(19) United States
(12) Patent Application Publication
(10) Pub. No.: US 2009/0275936 A1
(43) Pub. Date: Nov. 5, 2009

(54) SYSTEM AND METHOD FOR APPLYING THERAPY TO AN EYE USING ENERGY CONDUCTION

(76) Inventor: David Muller, Boston, MA (US)

Correspondence Address: NIXON PEABODY, LLP, 401 9TH STREET, NW, SUITE 900, WASHINGTON, DC, 20004-2128, US

(21) Appl. No.: 12/113,672

(22) Filed: May 1, 2008

(51) Int. Cl. A61F 9/007; A61B 18/18

(52) U.S. Cl. 606/33; 606/34; 606/41

(57) ABSTRACT

Thermokeratoplasty is applied to achieve a customized reshaping of a cornea, especially, for the treatment of astigmatism. Energy is applied to the cornea in a customized pattern using a specific configuration of two conductors. In one embodiment, an outer conductor and an outer conductor are separated by a gap. When a conducting element is applied to the corneal surface the area of the cornea at the periphery of the inner conductor is subject to an energy pattern with substantially the same shape and dimension as the gap between the inner and outer conductors. The inner and outer conductors may be positioned and shaped to form a gap having any desirable size and/or shape, including non-annular and asymmetrical shapes. The gap may be configured by altering the spatial relationships between the inner conductor and the outer conductor, by altering the size, shape, and/or position of the inner and/or outer conductors, or by forming one or more indentations or protrusions in or on the inner conductor and/or the outer conductor. Additionally or alternatively, energy is applied to the cornea in a customized pattern defined by a specific arrangement of one or more dielectric materials providing varying impedance.

### BACKGROUND OF THE INVENTION

1. Field of the Invention

The invention pertains generally to the field of keratoplasty and, more particularly, to a system and method for applying energy to an eye using energy conduction during thermokeratoplasty for the treatment of astigmatism or other eye disorders.

2. Description of Related Art

A variety of eye disorders, such as astigmatism, myopia, keratoconus, and hyperopia, involve abnormal shaping of the cornea. Keratoplasty reshapes the cornea to correct such disorders. For example, with astigmatism, there is an irregular curvature of the cornea, which is also referred to as a refractive error. Under normal circumstances, when light enters the eye, it refracts evenly, creating a clear view of the object. In contrast, with astigmatism, the eye may be shaped non-spherically, like a football or the back of a spoon. In this case, when light enters the eye it is refracted more in one direction than the other, allowing only part of the object to be in focus at one time. Objects at any distance can appear blurry and wavy. Astigmatism may also occur in combination with other refractive errors such as myopia (i.e. nearsightedness) and hyperopia (i.e. farsightedness).

One method for correcting astigmatism is by changing the shape of the cornea, for example, through refractive or laser eye surgery. Invasive surgical procedures, such as laser-assisted in-situ keratonomileusis (LASIK), may be employed, but typically require a healing period after surgery. Furthermore, such surgical procedures may involve complications, such as dry eye syndrome caused by the severing of corneal nerves.

Thermokeratoplasty, on the other hand, is a noninvasive procedure that may be used to correct the vision of persons who have disorders associated with abnormal shaping of the cornea. Thermokeratoplasty, for example, may be performed by applying electrical energy in the microwave or radio frequency (RF) band. In particular, microwave thermokeratoplasty may employ a near field microwave applicator to apply energy to the cornea and raise the corneal temperature. At about 60° C., the collagen fibers in the cornea shrink. The onset of shrinkage is rapid, and stresses resulting from this shrinkage reshape the corneal surface. Thus, application of energy in circular, ring-shaped patterns around the pupil may cause aspects of the cornea to flatten and improve vision in the eye. Although thermokeratoplasty has been identified as a technique for eye therapy, there is a need for a practical and improved system for applying thermokeratoplasty, particularly in a clinical setting.

### SUMMARY OF THE INVENTION

Embodiments according to aspects of the present invention relate generally to the field of keratoplasty and, more particularly, to a system and method for applying energy to an eye using energy conduction during thermokeratoplasty for the treatment of astigmatism or other eye disorders. In view of the asymmetrical and irregular shaping associated with eye disorders, such as astigmatism, the embodiments according to aspects of the invention are focused on also applying energy to an eye in asymmetrical and irregular patterns to treat such eye disorders.

For example, an energy conducting system for applying therapy to an eye includes an energy conducting element having a first conductor and a second conductor, where the first conductor and the second conductor extend to an application end and are separated by a gap. The energy conducting system includes a positioning system receives the energy conducting element and positions the distal end relative to a feature of an eye. Based in part on the position of the energy conducting element, the gap provides a pattern by which energy is delivered to the eye, where the pattern is non-annular and/or asymmetric with respect to the eye feature.

The energy conducting system also includes a positioning system that receives the energy conducting element. The gap provides a pattern for delivering energy to an eye when the positioning system positions the application end at the eye, the pattern being at least one of non-annular and asymmetric with respect to an eye feature.

In a further example, an embodiment relates to an energy conducting system for applying therapy to an eye, the energy conducting system including an outer conductor having an interior surface defining an interior passageway, and an inner conductor positioned within the interior passageway. The outer conductor and inner conductor define an application end positionable at an eye, with the outer conductor and inner conductor conducting energy to the eye via the application end. The inner conductor preferably has an exterior surface separated from the interior surface of the outer conductor by a gap, such that the gap has a varying thickness defined by more than one distance between the exterior surface of the inner conductor and the interior surface of the outer conductor. According to another embodiment, the inner conductor may have an exterior surface separated from the outer conductor by a non-annular (non-circular) gap. In a further alternative embodiment, at least one of the interior surface of the outer conductor and the inner conductor has a transverse profile having an indentation. In yet another alternative embodiment, at least one of the interior surface of the outer conductor and the inner conductor has a transverse profile having a protrusion.

Another embodiment relates to an energy conducting system for applying therapy to an eye, the energy conducting system including an outer conductor having an interior surface defining an interior passageway, an inner conductor positioned within the interior passageway, the inner conductor having an exterior surface separated from the interior surface of the outer conductor by a gap, wherein the outer conductor and inner conductor define an application end positionable at an eye, and one or more materials providing varying impedance, the one or more materials being applied, at the application end, to at least one of the outer conductor and the inner conductor, the outer conductor and inner conductor conducting energy to the eye via the application end according to the varying impedance.

Embodiments according to aspects of the invention are directed to a method for applying therapy to an eye with a conducting system comprising an energy conducting element including a first conductor and a second conductor, the first conductor and the second conductor extending to an application end and being separated by a gap, and a positioning system receiving the energy conducting element. A gap separating the first conductor and the second conductor is determined. The application end of the energy conducting element is positioned at an eye via the positioning system. An eye feature is reshaped by applying energy to the eye via the conducting element according to a pattern, the pattern being defined at least by the gap and the position of the application end relative to the eye and being at least one of non-annular and asymmetric with respect to the eye feature.

In addition, embodiments according to aspects of the invention relate to methods for applying therapy to an eye with a conducting assembly comprising an outer conductor having an interior surface defining a longitudinal interior passageway, and an inner conductor positioned within the interior passageway and having an exterior surface, wherein the outer conductor and inner conductor define an application end for conducting energy to the eye.

Another embodiment relates to a method including the steps of determining a gap separating exterior surface of the inner conductor from the interior surface of the outer conductor, the gap having a varying thickness defined by more than one distance between the inner conductor and the interior surface of the outer conductor, positioning the application end of the conducting assembly at an eye, and reshaping an eye feature by applying energy to the eye via the conducting element.

Still another embodiment relates to a method including the steps of determining a non-annular gap separating the exterior surface of the inner conductor from inner surface of the outer conductor, positioning the application end of the conducting assembly at an eye, and reshaping an eye feature by applying energy to the eye via the conducting element.

A further embodiment relates to a method including the steps of determining a gap separating the exterior surface of the inner conductor from inner surface of the outer conductor, wherein the gap is defined by at least one of the interior surface of the outer conductor and the inner conductor having a transverse profile having an indentation, positioning the application end of the conducting assembly at an eye, and reshaping an eye feature by applying energy to the eye via the conducting element.

Yet another embodiment relates to a method including the steps of determining a gap separating the exterior surface of the inner conductor from inner surface of the outer conductor, wherein the gap is defined by at least one of the interior surface of the outer conductor and the inner conductor having a transverse profile having a protrusion, positioning the application end of the conducting assembly at an eye, and reshaping an eye feature by applying energy to the eye via the conducting element.

The treatment of astigmatism with embodiments of the present invention is described herein to illustrate, by way of example, various aspects of the present invention. It is understood, however, that the embodiments are not limited to the treatment of astigmatism and may be applied in similar manner to treat other eye disorders, particularly those involving asymmetric or irregular shaping of the cornea.

These and other aspects of the present invention will become more apparent from the following detailed description of the preferred embodiments of the present invention when viewed in conjunction with the accompanying drawings.

### BRIEF DESCRIPTION OF THE DRAWINGS

FIG. 1 illustrates a cross-sectional view of an embodiment employing an electrical energy conducting element to reshape the cornea according to aspects of the present invention.

FIGS. 2A-2Q illustrate cross-sectional views of exemplary configurations of energy conducting elements having outer and inner conductors defining differently shaped gaps for applying energy in specific patterns to reshape a cornea according to aspects of the present invention.

FIGS. 3A-3B illustrate high resolution images of a cornea after energy has been applied.

FIGS. 3C-3D illustrate histology images of the cornea shown in FIGS. 3A-3B.

FIGS. 4A-4C illustrate perspective views of exemplary configurations of energy conducting elements having outer and inner conductors defining differently shaped gaps for applying energy in specific patterns to reshape a cornea according to aspects of the present invention.

FIGS. 5A-5B illustrate cross-sectional views of another embodiment employing an electrical energy conducting element to reshape a cornea according to aspects of the present invention.

FIG. 6 illustrates a cross-sectional view of another embodiment employing an electrical energy conducting element to reshape a cornea according to aspects of the present invention.

FIG. 7A-7L illustrate views of exemplary configurations of energy conducting elements that reshapes a cornea by applying energy in a pattern defined by a specific arrangement of varying thicknesses of a dielectric material providing varying impedance.

FIGS. 8A-8B illustrate views of exemplary configurations of energy conducting elements that reshapes a cornea by applying energy in a pattern defined by a specific arrangement of more than one dielectric material providing varying impedance.

FIGS. 9A-9B illustrate views of alternative shapes and configurations for conductors according to aspects of the present invention.

### DETAILED DESCRIPTION OF THE INVENTION

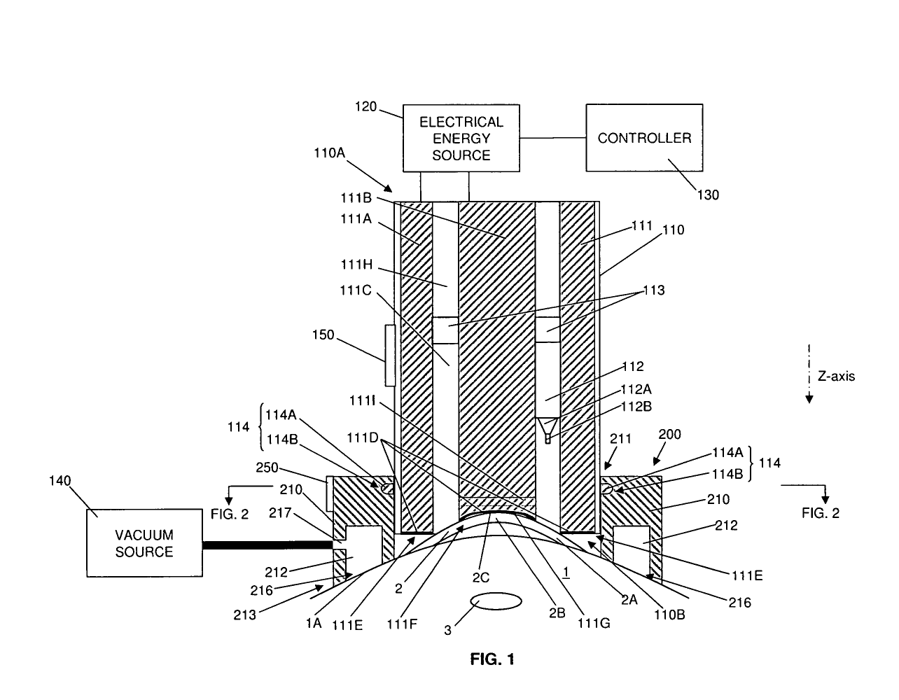

Referring to the cross-sectional view of FIG. 1, a system for applying energy to a cornea 2 of an eye 1 to achieve corrective reshaping of the cornea is illustrated. In particular, FIG. 1 shows an applicator 110 that includes an energy conducting element 111. The energy conducting element 111 extends through the applicator 110 from a proximal end 110A to a distal end 110B. An electrical energy source 120 is operably connected to the energy conducting element 111 at the proximal end 110A, for example, via conventional conducting cables. The electrical energy source 120 may include a microwave oscillator for generating microwave energy. For example, the oscillator may operate at a microwave frequency range of 500 MHz to 3000 MHz, and more specifically at a frequency of around 915 MHz which provides safe use of the energy conducting element 111. Although embodiments described herein may employ microwave frequencies, it is contemplated that any frequency, e.g., including microwave, radio-frequency (RF), etc., may be employed. For example, embodiments may employ radiation having, but not limited to, a frequency between 10 MHz and 300 GHz.

Operation of the energy source 120 causes energy to be conducted through the energy conducting element 111 to the distal end 110B. As such, the applicator 110 may be employed to apply energy to the cornea 2 of the eye 1 which is positioned at the distal end 110B. As shown further in FIG. 1, the distal end 110B is positioned over the cornea 2 by a positioning system 200. In general, the positioning system 200 provides support for the applicator 110 so that the energy conducting element 111 can be operated to deliver energy to targeted areas of the cornea 2. The positioning system 200 includes an attachment element 210 which receives the applicator 110. Meanwhile, the attachment element 210 can be fixed to a portion of the eye surface 1A, such as the area surrounding the cornea 2. The attachment element 210 situates the applicator 110 in a stable position for delivering energy to the cornea 2. When applying energy to the cornea 2 with an energy conducting element 111 as shown in FIG. 1, the energy conducting element 111 may be centered, for example, over the pupil 3, which is generally coincident with a center portion 2C of the cornea 2.

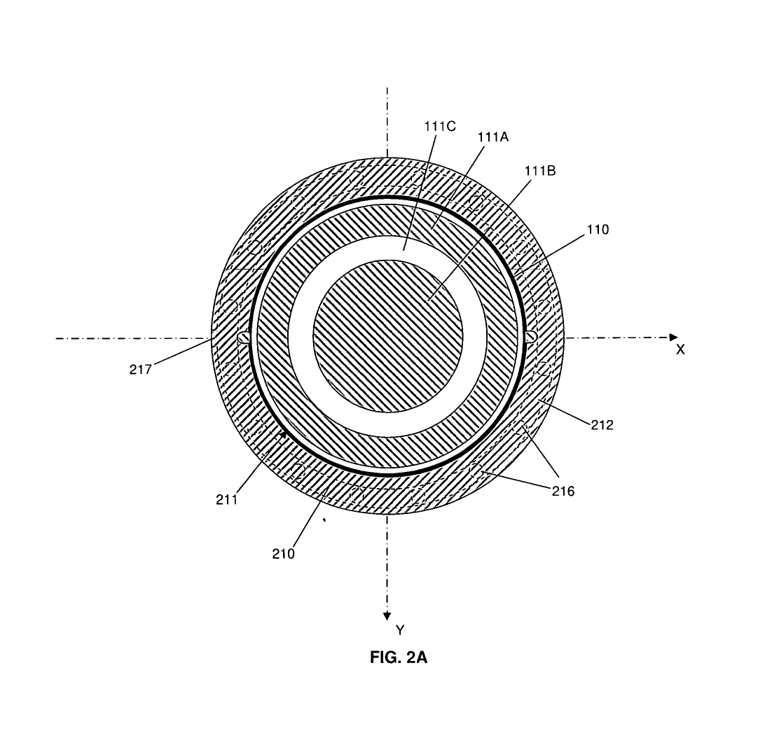

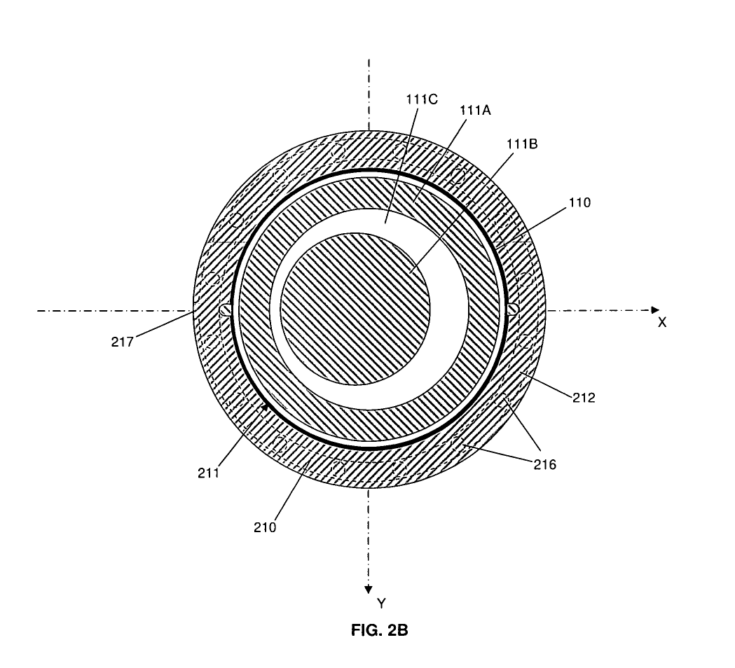

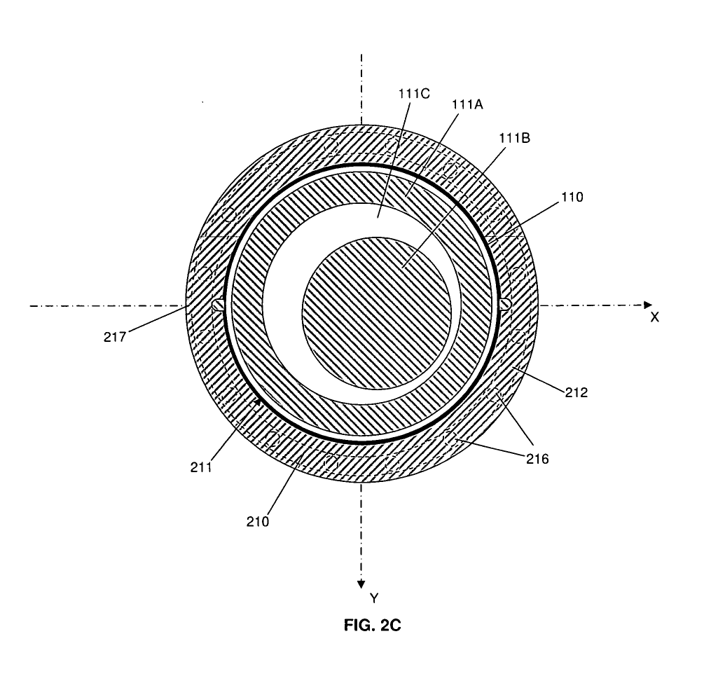

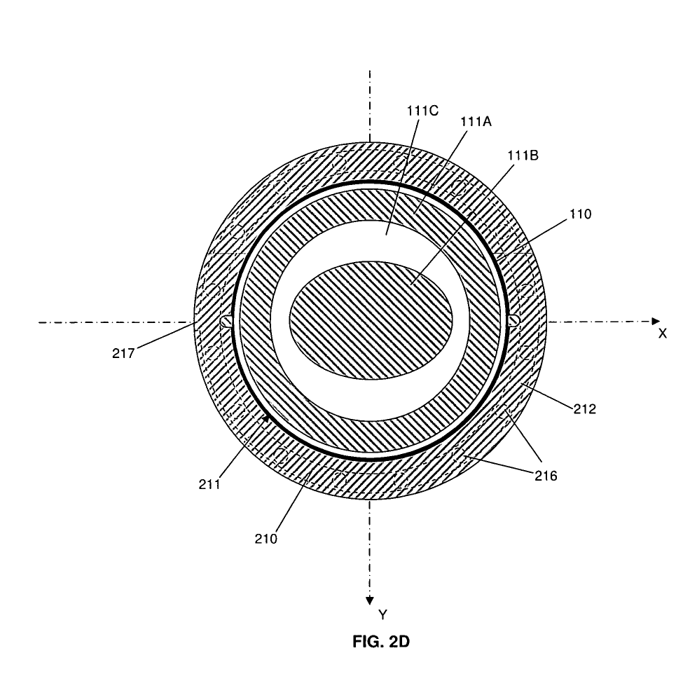

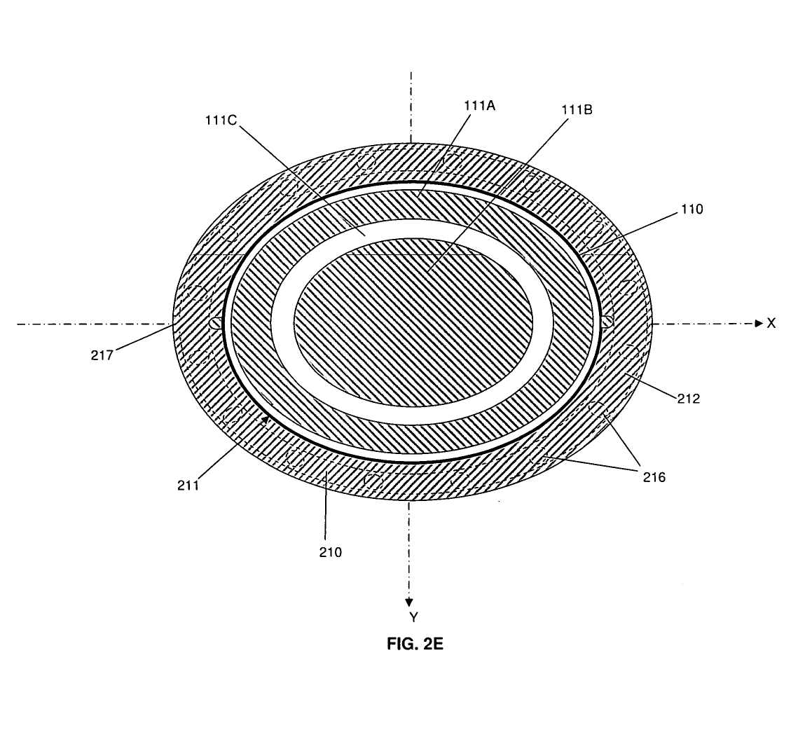

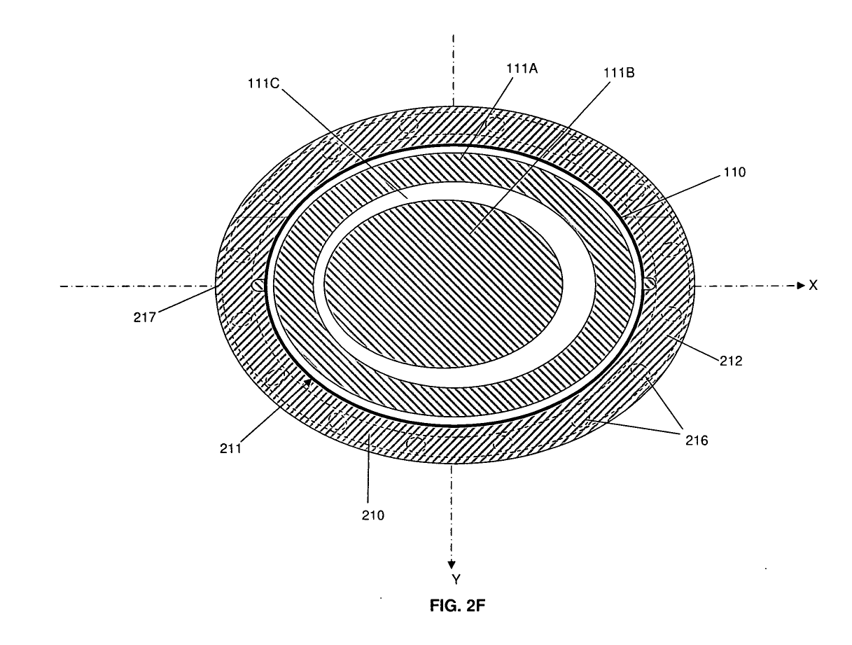

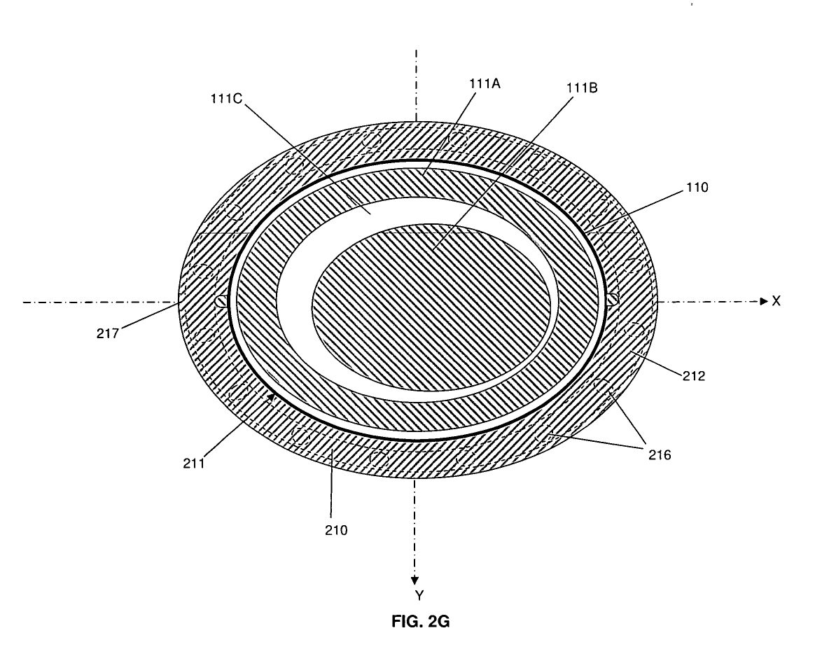

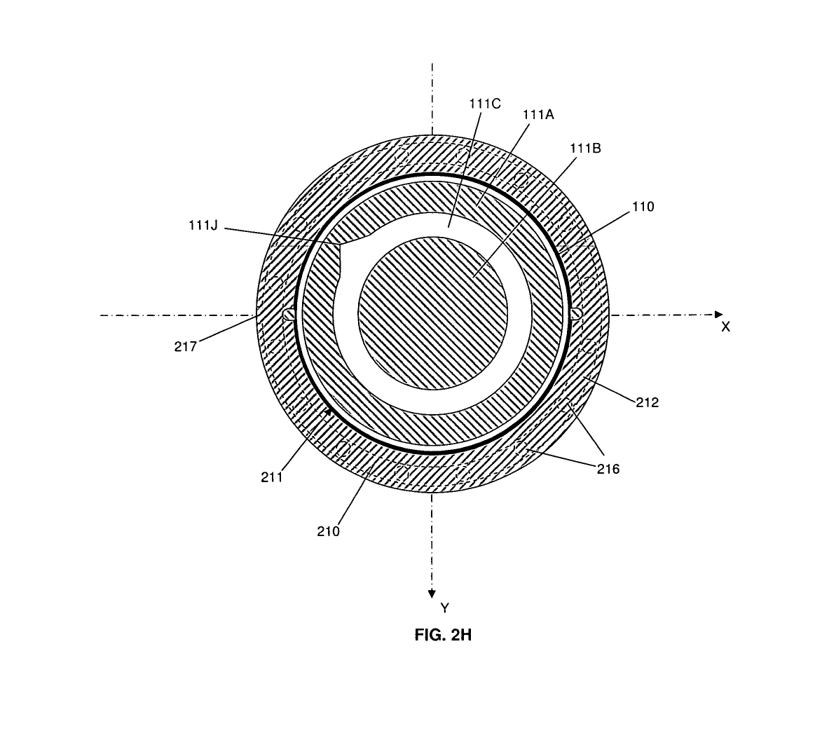

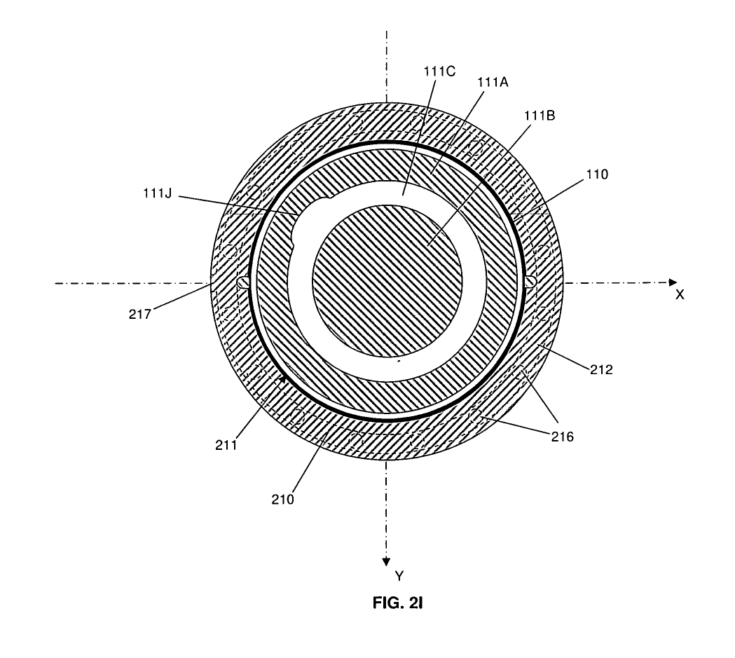

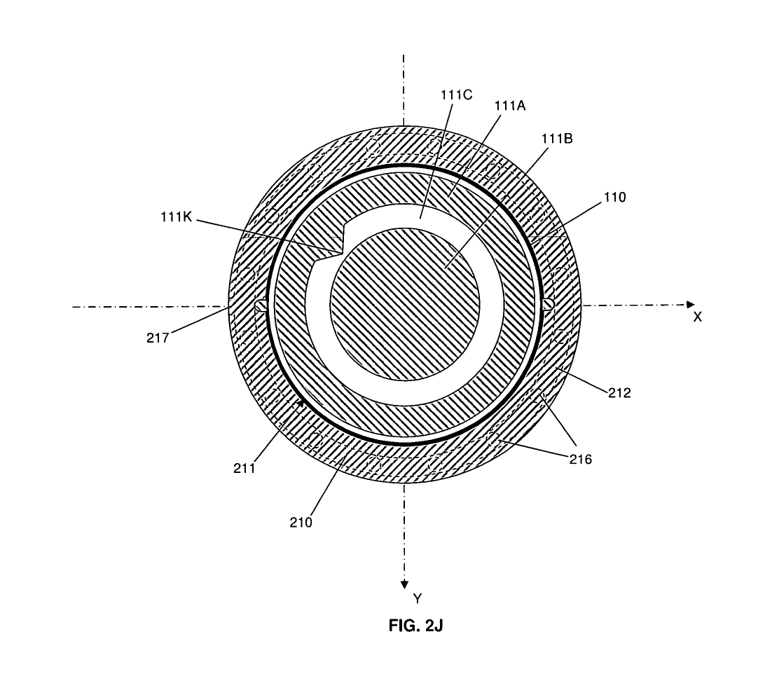

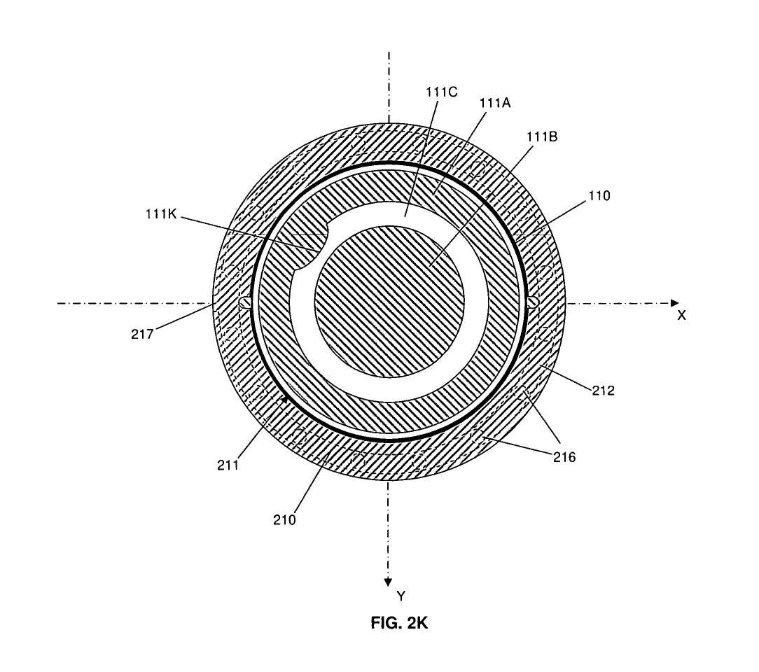

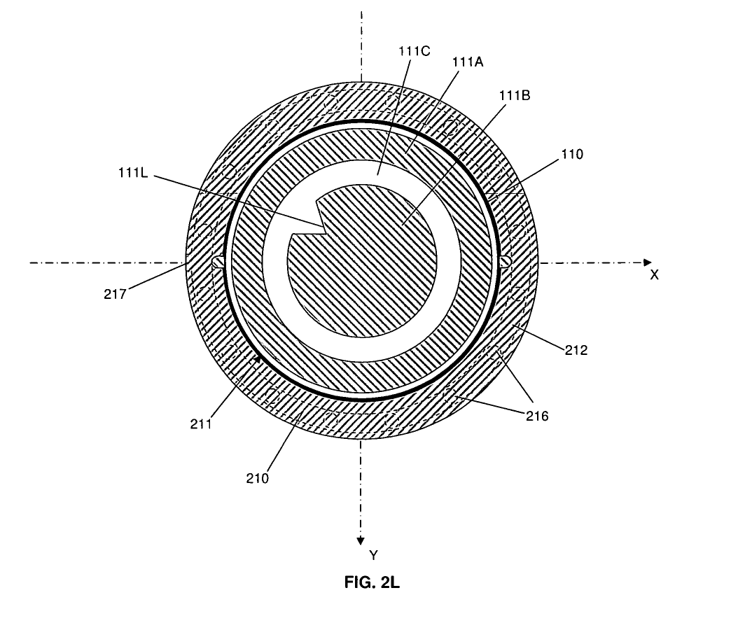

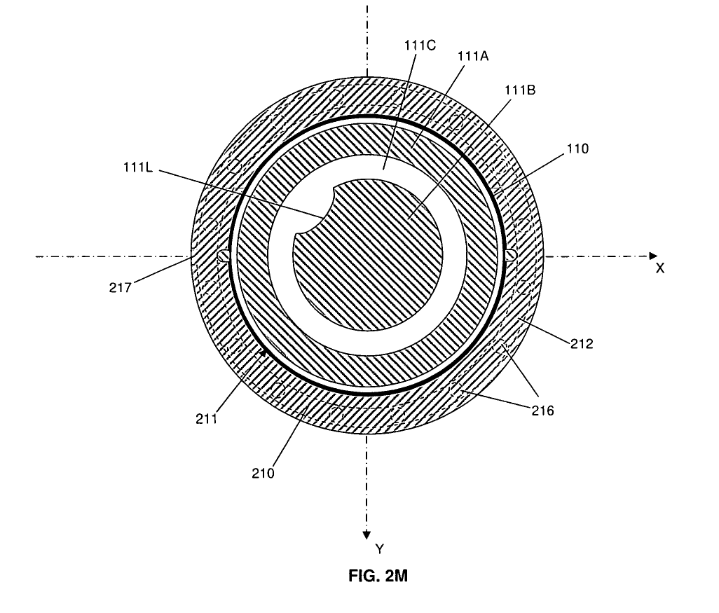

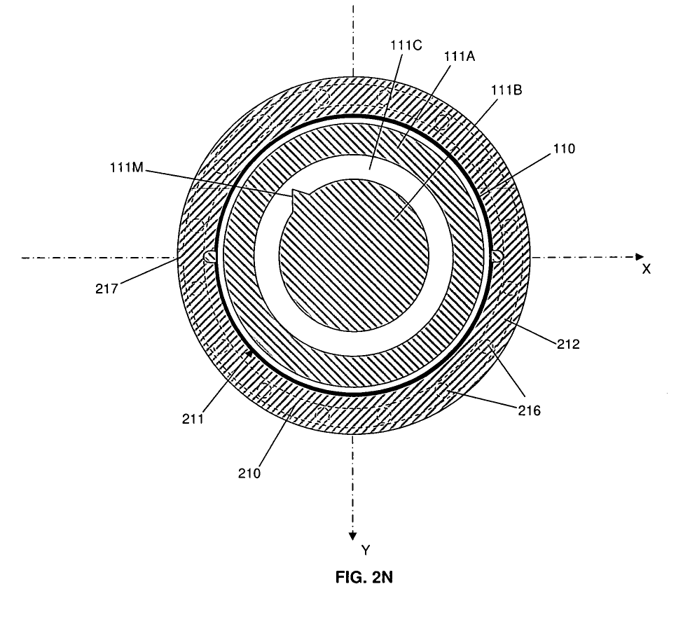

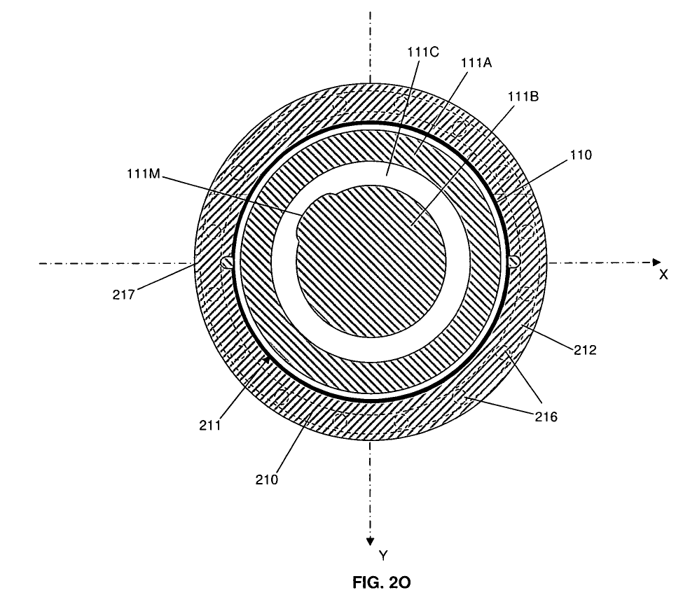

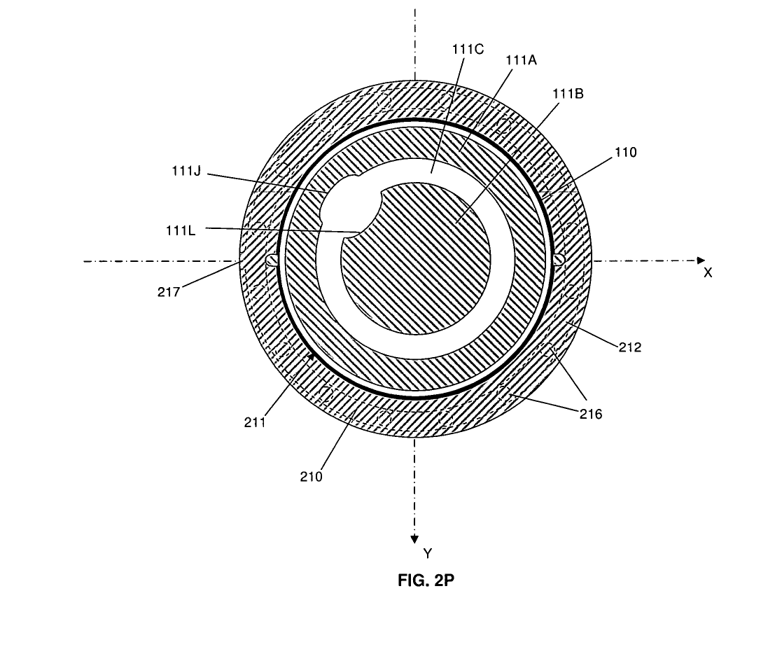

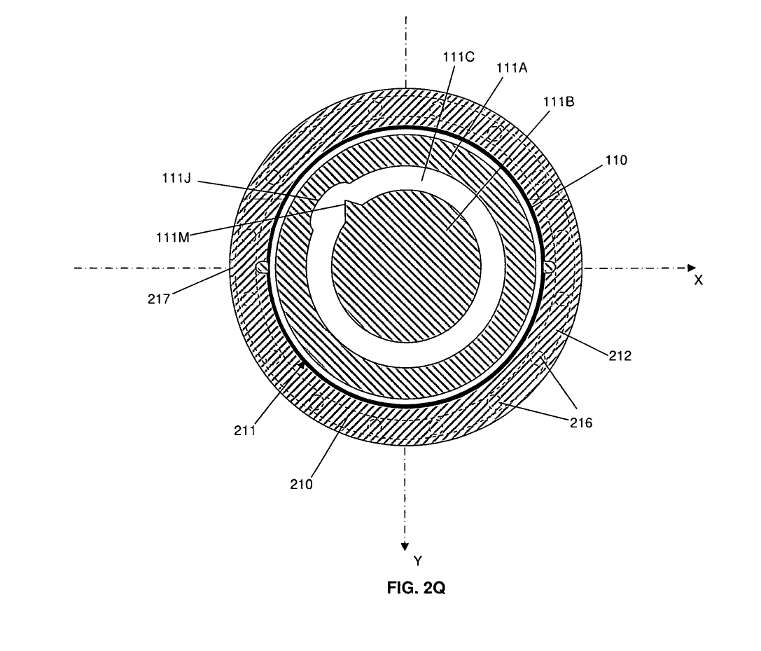

As shown in FIG. 1, the attachment element 210 of the positioning system 200 may have a central passageway 211 through which the applicator housing 110 can be received and the cornea 2 can be accessed. In some embodiments, for example, an outer dimension of the attachment element 210 may range from approximately 18 mm to 23 mm while an inner dimension may range from approximately 11 mm to 15 mm to accommodate aspects of the eye 1 and the cornea 2. The attachment element 210 may be attached to portions of the eye surface 1A by creating a vacuum connection with the eye surface 1A. As such, the attachment element 210 of FIG. 1 acts like a vacuum ring that includes an interior channel 212 which is operably connected to a vacuum source 140 via connection port 217. The attachment element 210 also includes a plurality of openings 216 which open the interior channel 212 to the eye surface 1A. The attachment element 210 may be formed from a biocompatible material such as a titanium alloy or the like. FIG. 2 illustrates a cross-sectional view of the attachment element 210, including the central passageway 211, the interior channel 212, the plurality of openings 216, and the connection port 217.

When the openings 216 are positioned in contact with the eye surface 1A and the vacuum source 140 is activated to create a near vacuum or low pressure within the interior channel 212, the openings 216 operate to suction the attachment element 210 and the eye surface 1A together. To promote sufficient suction between the eye surface 1A and the attachment element 210, the bottom surface 213 of the attachment element 210 may be contoured to fit the shape of the eye more closely. In one example, the vacuum source 140 may be a syringe, but the vacuum source 140 may be any manual or automated system that creates the appropriate amount of suction between the attachment element 210 and the eye surface 1A. Although the attachment element 210 can be stably attached to the eye surface 1A, the attachment element 210 can be detached by removing the vacuum source 140 and equalizing the pressure in the interior channel 212 with the exterior environment.

Once the applicator 110 is positioned by the positioning system 200, the energy conducting element 111 can deliver energy to targeted areas of collagen fibers in a mid-depth region 2B of the cornea 2 to shrink the collagen fibers according to a predetermined pattern and reshape the cornea 2 in a desired manner, thereby improving vision through the eye 1. For example, a contribution to the corneal reshaping comes from the contraction of the collagen fibrils found in the upper third of the corneal stroma, lying approximately 75-150 microns below the corneal, i.e., epithelial, surface 2A.

As further illustrated in FIG. 1, the electrical energy conducting element 111 may include two microwave conductors 111A and 111B, which extend from the proximal end 110A to the distal end 110B of the applicator 110. For example, as also illustrated in FIG. 2A, the conductor 111A may be a substantially cylindrical outer conductor, while the conductor 111B may be a substantially cylindrical inner conductor that extends through an inner passage extending through the outer conductor 111A. With the inner passage, the outer conductor 111A has a substantially tubular shape. The inner and the outer conductors 111A and 111B may be formed, for example, of aluminum, stainless steel, brass, copper, other metals, metal-coated plastic, or any other suitable conductive material. As described in detail below, aspects of the energy conducting element 111 may be shaped or contoured at the distal end 110B to promote desired shape changes with the cornea 2.

With the concentric arrangement of conductors 111A and 111B shown in FIG. 2A, a gap 111C is defined between the conductors 111A and 111B. The gap 111C extends from the proximal end 110A to the distal end 110B. A dielectric material 111H may be used in portions of the gap 111C to separate the conductors 111A and 111B. The distance of the gap 111C between conductors 111A and 111B determines the penetration depth of microwave energy into the cornea 2 according to established microwave field theory. Thus, the microwave conducting element 111 receives, at the proximal end 110A, the electrical energy generated by the electrical energy source 120, and directs microwave energy to the distal end 111B, where the cornea 2 is positioned in accordance with the positioning system 200.

The outer diameter of the inner conductor 111B is preferably larger than the pupil 3, over which the applicator 110 is centered. In general, the outer diameter of the inner conductor 111B may be selected to achieve an appropriate change in corneal shape, i.e. keratometry, induced by the exposure to microwave energy. The outer diameter of the inner conductor 111B determines the diameter across which the refractive change to the cornea 2 is made. When the energy conducting element is applied to the corneal surface 2A, the area of the cornea 2 at the periphery of the inner conductor 111B is subject to an energy pattern with substantially the same shape and dimension as the gap 111C between the two microwave conductors 111A and 111B.

Meanwhile, the inner diameter of the outer conductor 111A may be selected to achieve a desired gap between the conductors 111A and 111B. For example, the outer diameter of the inner conductor 111B ranges from about 4 mm to about 10 mm while the inner diameter of the outer conductor 111A ranges from about 4.1 mm to about 12 mm. In some systems, the gap 111C may be sufficiently small, e.g., in a range of about 0.1 mm to about 2.0 mm, to minimize exposure of the endothelial layer of the cornea (posterior surface) to elevated temperatures during the application of energy by the applicator 110.

A controller 130 may be employed to selectively apply the energy any number of times according to any predetermined or calculated sequence. The controller 130, for example, may be a programmable processing device, such as a conventional desktop computer, that executes software, or stored instructions. Controller 130 may also be a microprocessor device programmed in a known manner or any other device capable of controlling the process automatically or manually. In addition, the energy may be applied for any length of time. Furthermore, the magnitude of energy being applied may also be varied. Adjusting such parameters for the application of energy determines the extent of changes that are brought about within the cornea 2. Of course, the system attempts to limit the changes in the cornea 2 to an appropriate amount of shrinkage of collagen fibrils in a selected region. When delivering microwave energy to the cornea 2 with the applicator 110, the microwave energy may be applied with low power (of the order of 40 W) and in long pulse lengths (of the order of one second). However, other systems may apply the microwave energy in short pulses. In particular, it may be advantageous to apply the microwave energy with durations that are shorter than the thermal diffusion time in the cornea. For example, the microwave energy may be applied in pulses having a higher power in the range of 500 W to 3 KW and a pulse duration in the range of about 10 milliseconds to about one second.

Referring again to FIG. 1, at least a portion of each of the conductors 111A and 111B may be covered with an electrical insulator to minimize the concentration of electrical current in the area of contact between the corneal surface (epithelium) 2A and the conductors 111A and 111B. In some systems, the conductors 111A and 111B, or at least a portion thereof, may be coated with a material that can function both as an electrical insulator as well as a thermal conductor. Thus, a dielectric material 111D may be employed along the distal end 110B of the applicator 110, resulting in impedance that can protect the cornea 2 from electrical conduction current that would otherwise flow into the cornea 2 via conductors 111A and 111B.

Such current flow may cause unwanted temperature effects in the cornea 2 and interfere with achieving a maximum temperature within the collagen fibrils in a mid-depth region 2B of the cornea 2. Accordingly, the dielectric material 111D is positioned between the conductors 111A and 111B and the cornea 2. In particular, as shown in FIG. 1, the distal ends 111E and 111F of the conductors 111A and 111B include a dielectric material 111D. The dielectric material 111D may be sufficiently thin to minimize interference with microwave emissions and thick enough to prevent superficial deposition of electrical energy by flow of conduction current. For example, the dielectric material 111D may be a biocompatible material, such as Teflon® fluoropolymer resin, deposited to a thickness of about 0.002 inches. Other suitable dielectric materials include, for example, Kapton® polymide film.

In general, an interposing layer, such as the dielectric material 111D, may be employed between the conductors 111A and 111B and the cornea 2 as long as the interposing layer does not substantially interfere with the strength and penetration of the microwave radiation field in the cornea 2 and does not prevent sufficient penetration of the microwave field and generation of a desired energy pattern in the cornea 2. Alternatively, the dielectric material 111D may be omitted and electrical energy in the microwave or radio frequency (RF) band may be applied directly.

During operation, the distal end 110B of the applicator 110 as shown in FIG. 1 is positioned by the positioning system 200 at the corneal surface 2A. Preferably, the energy conducting element 111 makes direct contact with the corneal surface 2A. As such, the conductors 111A and 111B are positioned at the corneal surface 2A. The positioning of the conductors 111A and 111B helps ensure that the pattern of microwave energy delivered to the corneal tissue has substantially the same shape and dimension as the gap 111C between the two microwave conductors 111A and 111B.

As shown in FIG. 1, the applicator 110 may also employ a coolant system 112 that selectively applies coolant to the corneal surface to minimize heat-related, damage to the corneal surface 2A during thermokeratoplasty and to determine the depth of energy delivered below the corneal surface 2A to the mid-depth region 2B. Such a coolant system enables the energy conducting element 111 to be placed into direct contact with the corneal surface 2A without causing energy-related damage. In some embodiments, the coolant may also be applied after the application of energy to preserve, or “set,” the desired shape changes by eliminating further energy-induced changes and preventing further changes to the new corneal shape. Examples of such a coolant system are described in U.S. application Ser. No. 11/898,189, filed Sep. 10, 2007, the contents of which are entirely incorporated herein by reference. For example, the coolant delivery system 112 as well as a coolant supply 113 may be positioned within the gap 111C. Although FIG. 1 may illustrate one coolant delivery system 112, the applicator 110 may include a plurality of coolant delivery systems 112 arranged circumferentially within the gap 111C. The coolant supply 113 may be a container that fits within the gap 111C, with the coolant delivery element 112 having a nozzle structure 112A extending downwardly from the coolant supply 113 and an opening 112B directed toward the distal end 110B. The coolant may be a liquid cryogen, such as tetrafluorothane. Alternatively, the coolant may be a cool gas having a sufficiently low temperature to remove energy at a desired rate, such as nitrogen gas, e.g., blowoff from a liquid nitrogen source.

In some embodiments, the coolant system 112 is operated, for example, with the controller 130 to deliver pulses of coolant in combination with the delivery of energy to the cornea 2. Advantageously, applying the coolant in the form of pulses can help prevent the creation of a fluid layer between the conductors 111A and 111B and the corneal surface 2A. In particular, the short pulses of coolant may evaporate from the corneal surface 2A or may be removed, for example, by a vacuum (not shown) before the application of the microwave energy. Rather than creating an annular energy pattern according to the dimensions of the conductors 111A and 111B, the presence of such a fluid layer may disadvantageously cause a less desirable circle-shaped microwave energy pattern in the cornea 2 with a diameter less than that of the inner conductor 111B. Therefore, to achieve a desired microwave pattern in some embodiments, a flow of coolant or a coolant layer does not exist over the corneal surface 2A during the application of energy to the cornea 2. To further minimize the presence of a fluid layer, as described previously, the coolant may actually be a cool gas, rather than a liquid coolant.

Of course, in other embodiments, a flow of coolant or a coolant layer can be employed, but such a layer or flow is generally controlled to promote the application of a predictable microwave pattern. Additionally or alternatively, heat sinks may also be employed to direct heat away from the corneal surface 2A and reduce the temperature at the surface 2A.

In addition to the characteristics described above with reference to FIG. 2A, FIGS. 2A-2Q are cross-sectional illustrations of various configurations of the energy conducting systems of the invention. To treat astigmatism, for example, the spatial relationships between the outer conductor and the inner conductor may be altered to form a gap that is suitable to treat the specific type of astigmatism exhibited by the patient. As each patient is different, non-annular (non-circular) and/or asymmetrical gaps may be needed to effectively treat the patient's astigmatism.

For example, FIG. 2A illustrates a cross-sectional view of an energy conducting system including, for example, an outer conductor 111A having an interior surface defining an interior passageway, and an inner conductor 111B positioned within the interior passageway. The inner conductor 111B has an exterior surface separated from the interior surface of the outer conductor 111A by a gap 111C. In the illustration of FIG. 2A, the gap 111C is substantially annular, and is substantially symmetrical relative to both the vertical Y-axis, and the horizontal X-axis. In addition, the gap 111C in FIG. 2A has substantially the same thickness between the inner surface of outer conductor 111A and the outer surface of inner conductor 111C.

However, to treat astigmatism or other eye disorder, the gap 111C may have to be irregularly shaped, e.g., asymmetric and/or non-annular. As discussed previously, the shape of the gap 111C determines the pattern by which energy is delivered to the cornea 2 and selective shrinkage of the corneal fibers is achieved. For example, FIG. 2B illustrates an embodiment in which gap 111C has a varying thickness defined by more than one distance between the exterior surface of the inner conductor 111B and the interior surface of the outer conductor 111A. In FIG. 2B, even though inner conductor 111B is substantially cylindrical, the central axis of inner conductor 111B is not positioned in alignment with the central axis of outer conductor 111A. The offset between the central axis of inner conductor 111B and the central axis of outer conductor 111A results in gap 111C being non-annular. In the embodiment shown in FIG. 2B, gap 111C has a wider thickness on one side of inner conductor 111B in FIG. 2B than on the opposing side of inner conductor 111B.

In addition, inner conductor 111B may be adjustably movable relative to outer conductor 111A. To illustrate this, the embodiment shown in FIG. 2B and the embodiment shown in FIG. 2C illustrates two exemplary positions of inner conductor 111B relative to outer conductor 111A. In both figures, inner conductor 111B is substantially cylindrical, and gap 111C is non-annular and has a varying thickness. However, the position of inner conductor 111B relative to outer conductor 111A has been adjusted. The position of inner conductor 111B relative to outer conductor 111A may be modified as needed to form a gap of an appropriate size and shape to treat a patient's specific astigmatism. In some embodiments, an adjustable fixation system may be employed, at proximal end 110A for example, to fix the position of the inner conductor 111B relative to the outer conductor 111A once the position has been modified.

FIG. 2D illustrates an alternative exemplary configuration in which inner conductor 111B is not cylindrically shaped. Instead, inner conductor 111B is substantially elliptical. As a result, gap 111C is non-annularly shaped and has a varying thickness. Thus, it is possible to achieve a non-annular gap 111C without requiring the center of a cylindrical inner conductor 111B to be offset relative to the center of a cylindrical outer conductor 111A as shown in FIGS. 2B and 2C.

FIGS. 2E-2G illustrate an embodiment in which both inner conductor 111B and outer conductor 111A are non-cylindrically shaped. Specifically, in these figures, both inner conductor 111B and outer conductor 111A are elliptically shaped. In FIG. 2E, gap 111C is non-annularly shaped, yet still has a substantially even thickness between the inner surface of outer conductor 111A and the outer surface of inner conductor 111B. Thus, by using inner conductors and outer conductors that are similarly shaped, it is possible to alter the shape of the gap without necessarily forming a gap that has varying thicknesses.

FIG. 2F illustrate an alternative configuration of the embodiment shown in FIG. 2E, wherein the central axis of inner conductor 111B is offset from the central axis of outer conductor 111A. As a result, gap 111C no longer has a substantially even thickness, and instead has a varying thickness. In addition, as described above, and as is illustrated by a comparison between FIGS. 2F and 2G, inner conductor 111B may be adjustably movable relative to outer conductor 111A, regardless of the relative shapes of inner conductor 111B and outer conductor 111A.

In addition, it may be desirable for gap 111C to be asymmetrically shaped to treat different specific conditions. For example, the embodiment of FIG. 2F illustrates a configuration in which gap 111C is substantially symmetrical relative to the horizontal X-axis, but asymmetrical relative to the vertical Y-axis. In contrast, the configuration shown in FIG. 2G results in gap 111C being asymmetrical relative to both the vertical Y-axis and the horizontal X-axis. Thus, by adjusting the position of inner conductor 111B relative to outer conductor 111A, the symmetry or asymmetry of gap 111C relative to the horizontal or vertical axes may be controlled.

According to a further embodiment, one or both of inner conductor 111B and outer conductor 111A may be irregularly shaped. The shape of inner conductor 111B and outer conductor 111A may be altered as desired to create a customized shape and/or size of gap 111C.

For example, as is shown in FIGS. 2H-2I, one or more outer conductor indentations 111J may be formed in outer conductor 111A. FIG. 2H shows an exemplary configuration in which indentation 111J is a notch. FIG. 2I shows an exemplary alternative configuration in which indentation 111J is curved. Alternatively, as is shown in FIGS. 2J-2K, outer conductor 111A is shown with a protrusion 111K that extends into gap 111C. FIG. 2J shows a protrusion 111K that has an angled shape, while FIG. 2K shows a protrusion 111K that has a curved shape.

FIGS. 2L-2O illustrate an embodiment in which the shape of inner conductor 111B is customized. For example, FIGS. 2L-2M illustrate exemplary configurations in which indentations 111L are formed in inner conductor 111B. Indentation 111L is a notch in FIG. 2L, and is curved in FIG. 2M. Alternatively, FIGS. 2N-2O illustrate exemplary embodiments in which a protrusion 111M is formed on inner conductor 111B. Protrusion 111M is an angled shape in FIG. 2N, and is a curved shape in FIG. 2O.

In addition, FIGS. 2P-2Q illustrate exemplary configurations in which indentations and/or protrusions are formed into, or onto, both inner conductor 111B and outer conductor 111A. In FIG. 2P, a curved indentation 111J is formed into outer conductor 111A and a curved indentation 111L is formed into inner conductor 111B. In FIG. 2Q, a curved indentation 111J is formed into outer conductor 111A, and an angled protrusion 111M is formed onto inner conductor 111B. Thus, one or more indentation may be used in combination with one or more protrusions, as desired.

In this regard, any suitable shape or size of indentations and/or protrusions may be formed into, or onto, either of the outer conductor or the inner conductor. In addition, multiple indentations and/or protrusions may be formed into, or onto, either of the inner conductor and/or the outer conductor may be used, as desired. In addition, the positioning of the indentations and protrusions shown in the figures was arbitrary, and one or more indentations or protrusions may be formed into, or onto, either of the outer conductor or the inner conductor, in any suitable position relative to gap 111C, and to any of inner conductor 111B, outer conductor 111A, or any other indentations or protrusions. By forming indentations and/or protrusions into, or onto, the inner conductor and/or the outer conductor, the size and shape of the gap may be customized and controlled in a novel and advantageous manner.

FIG. 2R illustrates another embodiment in which the outer conductor 111A and the inner conductor 111B delivers energy in a non-annular and asymmetric pattern to the eye. In particular, the outer conductor 111A includes one or more intervals 111N that segments the outer conductor 111A to have a non-continuous shape. In addition, the inner conductor 111B includes one or more intervals 111O that segments the inner conductor 111B. As shown in FIG. 2R, the intervals 111N are defined by spaces that extend radially through the wall of the outer conductor 111A at the distal end 110B. Meanwhile, the interval 111O is defined by a space that extend through the inner conductor 111B at the distal end 110B. Energy is conducted from areas of the gap 111C where there are opposing sections of outer conductor 111A and inner conductor 111B. In other words, no energy is conducted from areas of the gap 111C that are positioned between the intervals 111N and the inner conductor 111B or between the intervals 111O and the outer conductor 111A. Thus, whereas FIG. 2A illustrates an embodiment that delivers energy in a continuous annular pattern defined by the annular gap 111C, the selected positioning of intervals 111N and 111O creates a segmented and non-continuous pattern in the embodiment of FIG. 2R. Of course, the embodiment shown in FIG. 2R is provided merely as an example, and any number of intervals 111N and 111O having any size may be employed to achieve a non-annular and/or asymmetric pattern. Moreover, alternative embodiments may employ just the intervals 111N or just the intervals 111O, rather than both.

In sum, FIGS. 2B-2R illustrate embodiments in which the energy conducting element 111 includes an outer conductor 111A and an inner conductor 111B that are not cylindrical and/or concentric with respect to each other. As such, these embodiments can apply energy to an eye in asymmetrical, non-annular, and/or other irregular patterns to treat eye disorders, such as astigmatism. Other embodiments, however, are able to achieve asymmetrical and irregular patterns by, additionally or alternatively, modifying other aspects of the energy conducting element 111. As described above, with reference to FIG. 1, a dielectric material 111D may be employed along the distal end 110B of the applicator 110 to protect the cornea 2 from electrical conduction current that would otherwise flow into the cornea 2 via conductors 111A and 111B. It has been discovered that applying a dielectric material 111D, such as Kapton® polymide film, having a varying thickness along the distal end 111E of the outer conductor 111A and/or the distal end 111F of the inner conductor 111B provides another technique for determining the pattern of energy delivered by the energy conducting element 111 to the cornea 2.

The presence of a dielectric material 111D results in impedance that affects the delivery of energy from the energy conducting element 111. As such, changing the application of the dielectric material 111D changes the impedance characteristics of the energy conducting element 111. For example, a thicker layer of a given dielectric material 111D provides greater impedance and minimizes conductivity through the dielectric layer, while a thinner layer of the same dielectric material 111D provides less impedance and may permit an amount of conductivity through the layer. Therefore, rather than applying a substantially uniform layer of a given dielectric material 111D, embodiments may apply the dielectric material 111D in a layer of varying thickness, where energy is substantially prevented from passing through thicker portions of the dielectric layer but can pass through the thinner portions. Accordingly, the thicker portions may be arranged in combination with the thinner portions to create a pattern that blocks the delivery of energy to selected portions of the eye while allowing delivery to other portions. As used herein, reference to “thicker portions” indicates application of a dielectric material that has sufficient impedance to substantially prevent energy from being conducted through the layer, while reference to “thinner portions” indicates application of a dielectric material that has sufficiently low impedance to permit energy to pass through the layer to the eye. As described further below, the actual dimensions of the thicker layer and the thinner layer depend on the material from which the layers are formed. Different materials may require the application of different thicknesses to achieve a given impedance. It is also contemplated that the dimensions of the thinner portions may be reduced to an extreme where the reduction results in the absence of any dielectric material. It is further contemplated that the thicker portion and/or thinner portion may each have a non-uniform thickness. Thus, the impedence across the thinner section may also vary.

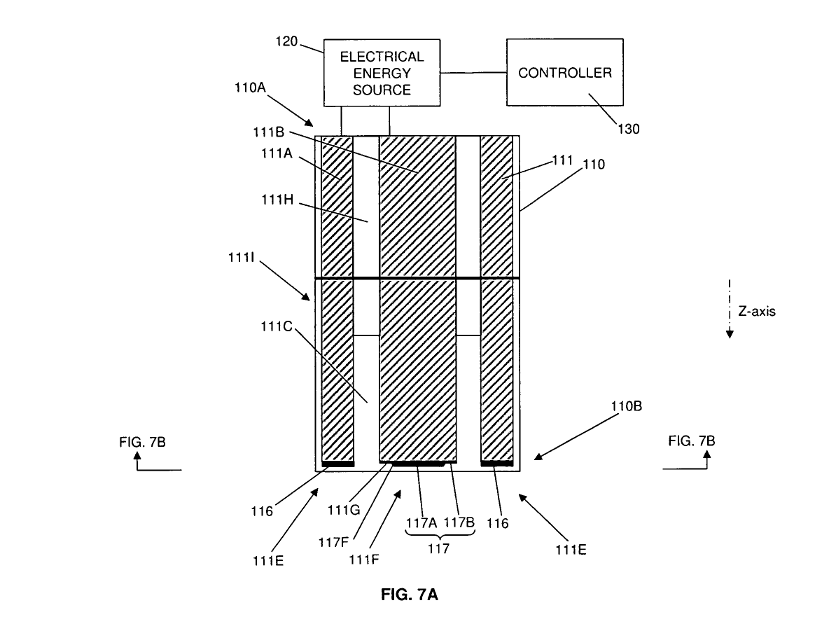

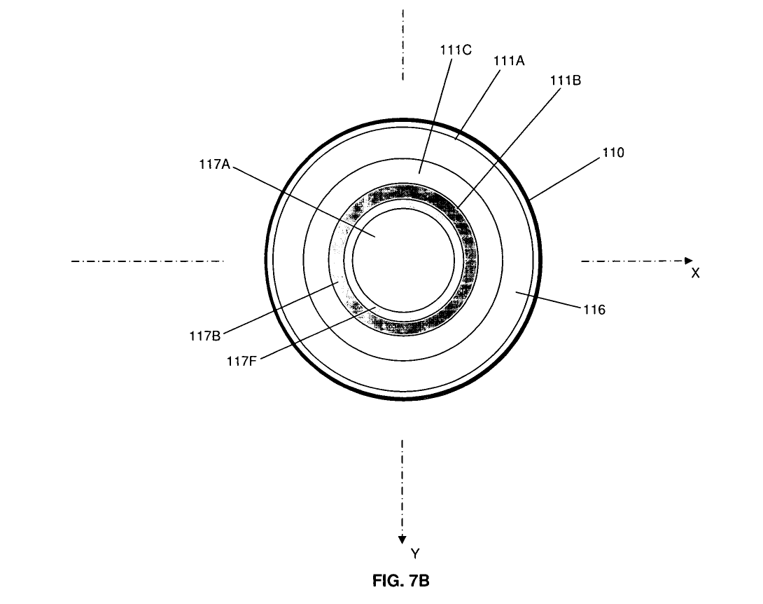

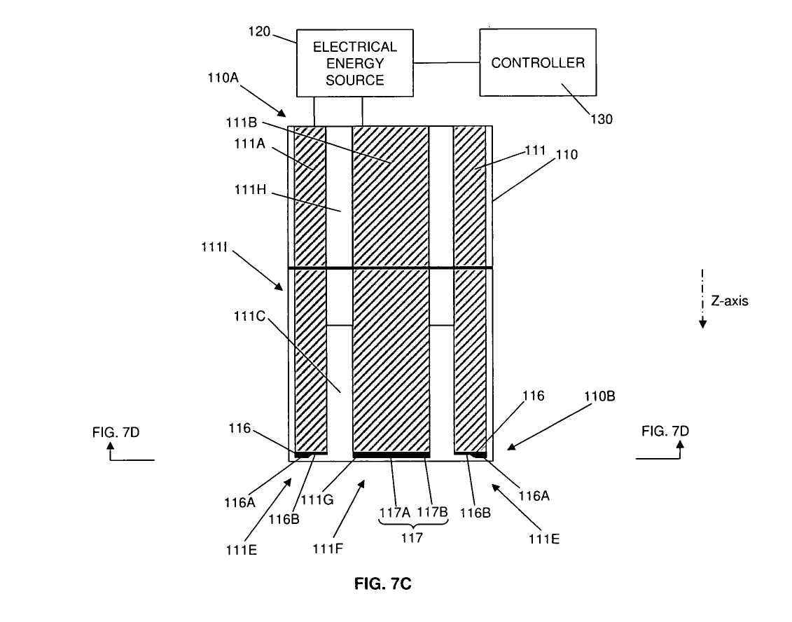

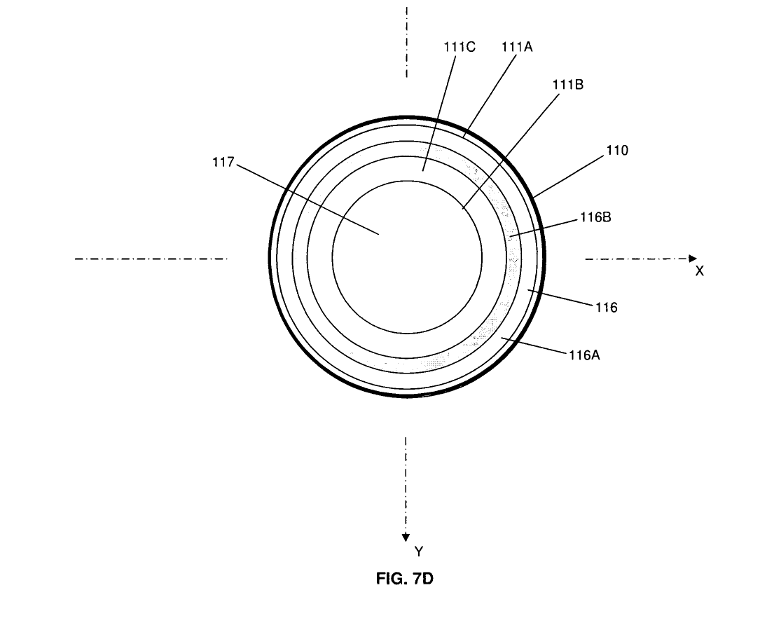

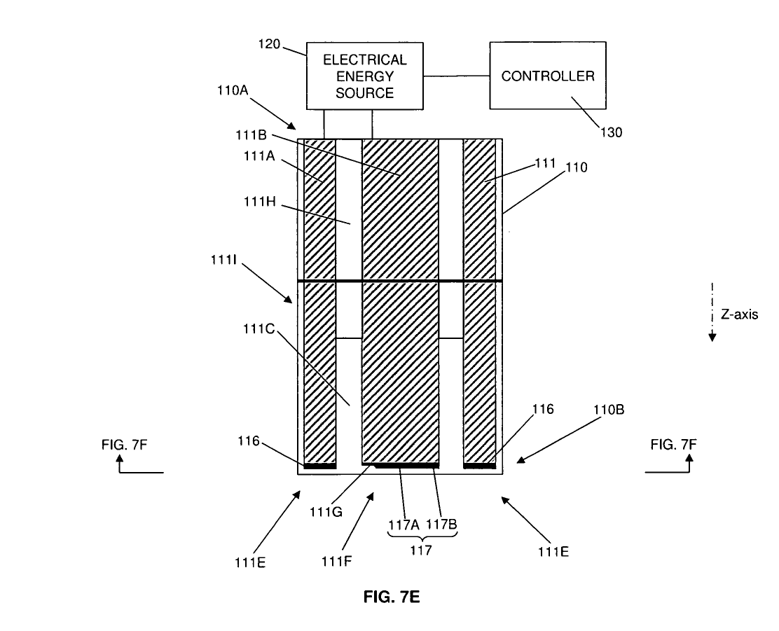

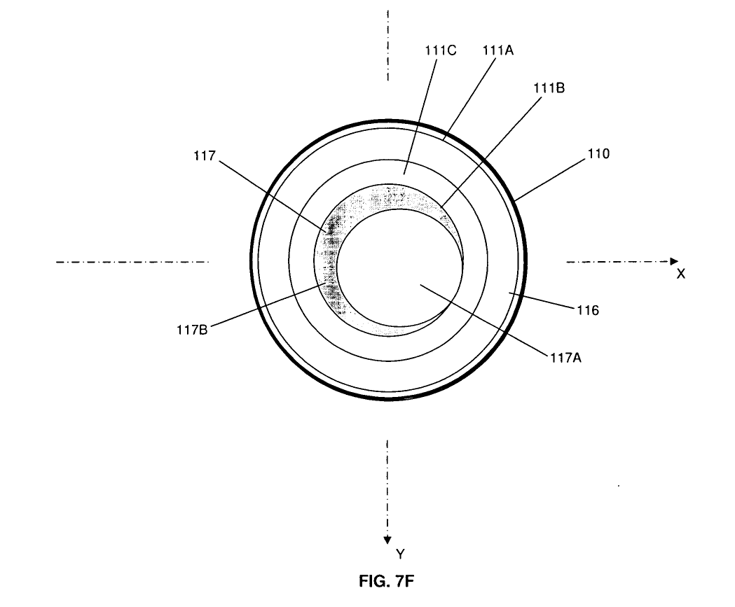

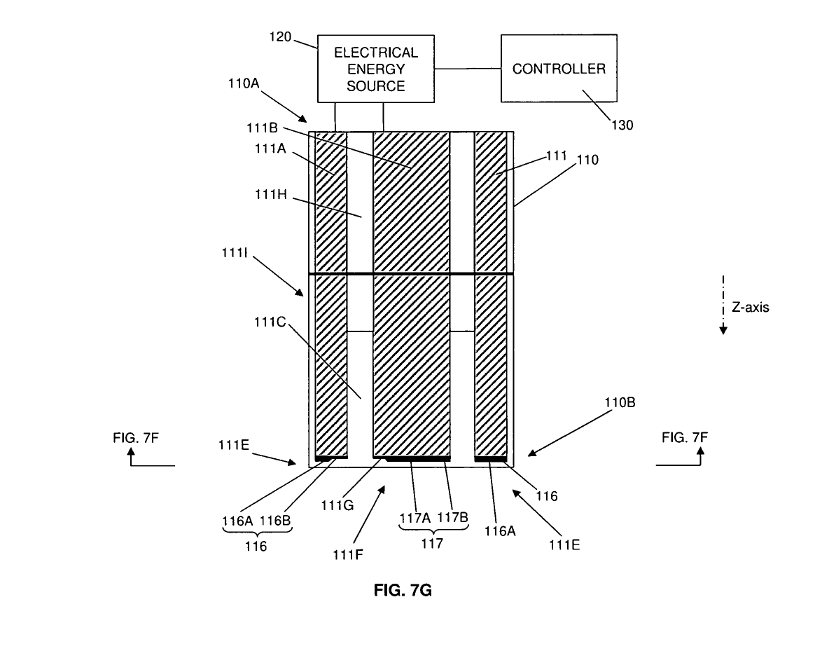

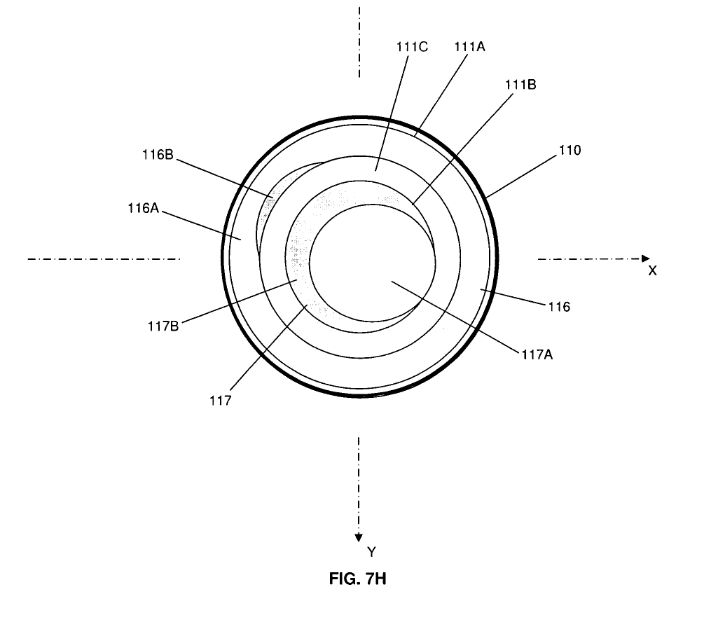

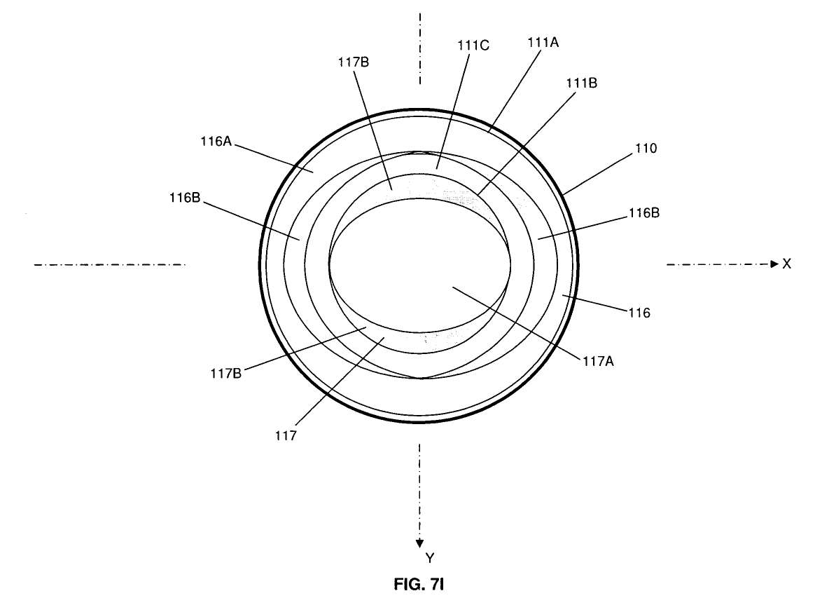

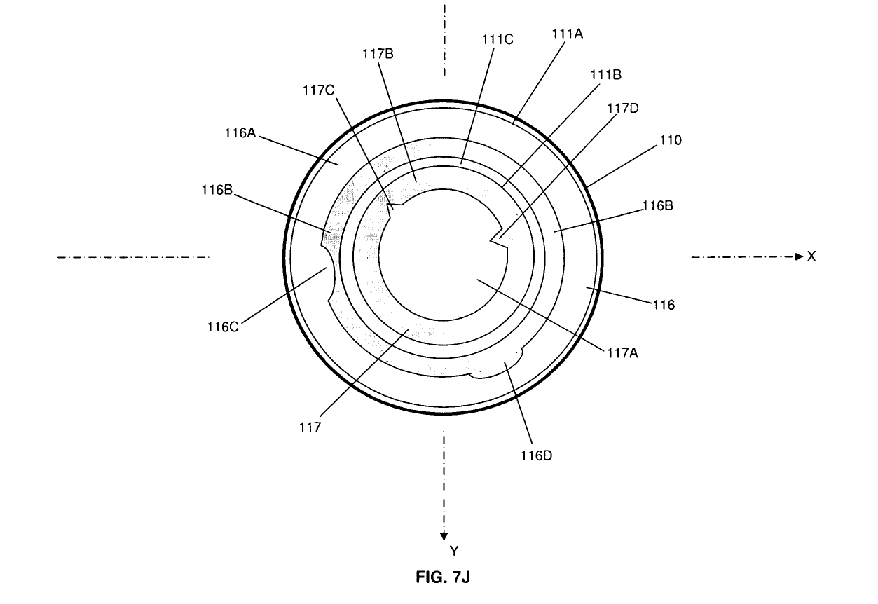

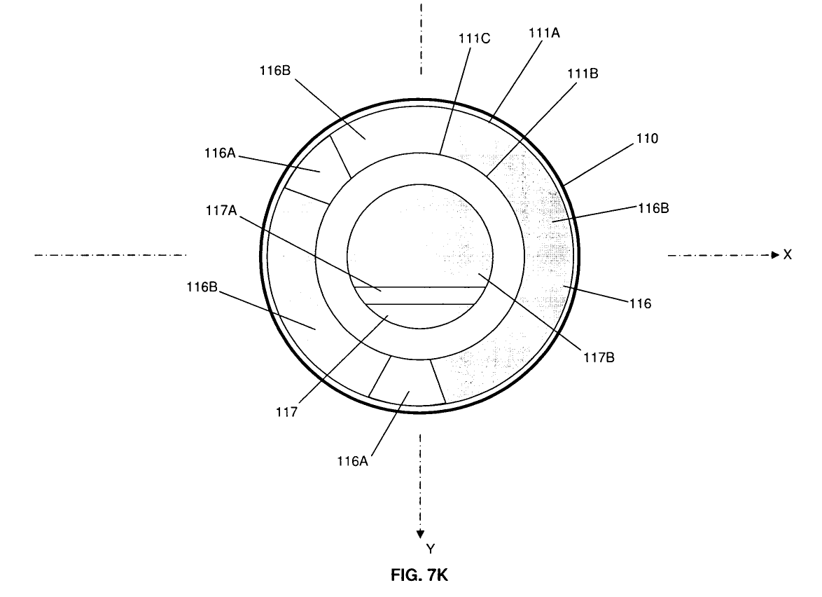

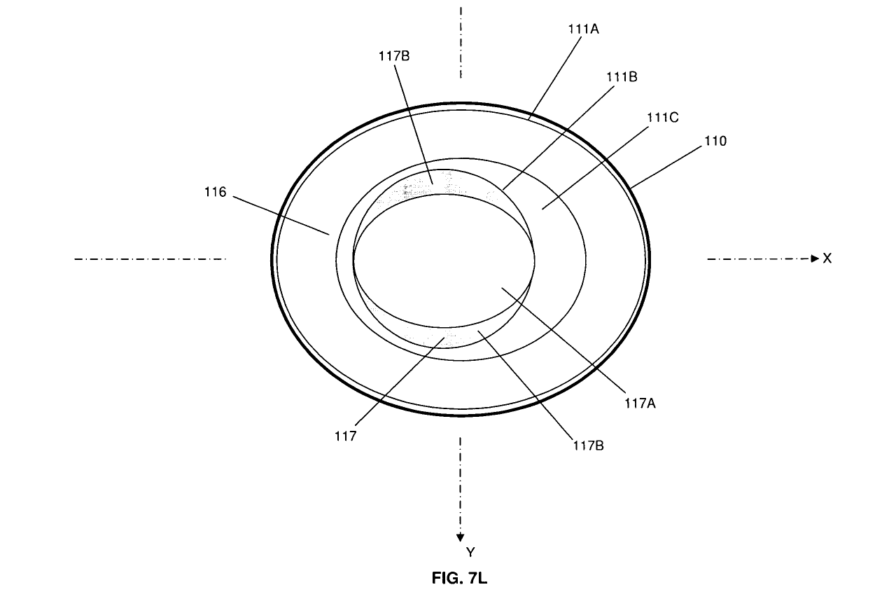

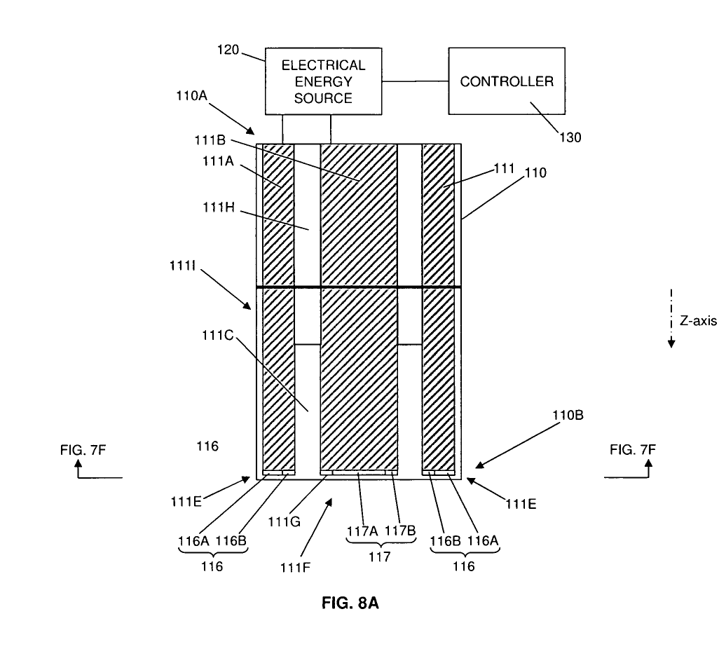

FIG. 7A illustrates an applicator 110 including an energy conducting element 111 that is similar in many respects to the applicator 110 shown in FIG. 1. In the embodiment of FIG. 7A, however, a dielectric material 111D is applied is applied to the energy conducting element 111 in varying thicknesses. In particular, a dielectric layer 116 is applied to the distal end 111E of the outer conductor 111A and a dielectric layer 117 is applied to the distal end 111F of the inner conductor 111B. In addition, the dielectric layer 117 includes a thicker portion 117A and a thinner portion 117B. FIG. 7B shows a view of the surfaces of the dielectric layers 116 and 117 as indicated in FIG. 7A. As FIG. 7B illustrates, the thicker portion 117A defines a substantially circular shape that is generally concentric with the inner conductor 111B. Meanwhile, the thinner portion 117B defines a substantially annular shape that is generally concentric with the circular layer 117A and the inner conductor 111B. For example, as shown in FIG. 7B, the diameter of the substantially cylindrical inner conductor 111B may be approximately 7 mm, while the diameter of the circular thicker portion 117A may be about 5 mm and the annular thickness of the portion 117B may be about 2 mm. When energy from the energy source 120 is conducted through the energy conducting element 111, energy can pass through the layer 117B, but not through the layer 117A. As FIGS. 7A and 7B illustrate, the dielectric layer 117 may also include a contoured, beveled, or sloped surface 117F to provide a smoother or gradual transition between portions 117A and 117B. Although not always shown in the figures, it is understood that any of the embodiments described herein may employ such a surface between portions having different thicknesses. In addition, to make aspects of the dielectric layers 117 and 118 clearer in FIG. 7A, the shape of the surface 111G at the distal end 111F is shown to be planar, but it is understood that the surface 111G may be contoured or curved as described herein.

As described above, when the embodiment of FIG. 1 is employed, the area of the cornea 2 at the periphery of the inner conductor 111B is subject to an energy pattern with substantially the same shape and dimension as the gap 111C between the two microwave conductors 111A and 111B. For example, a dielectric material 111D of sufficient thickness may be employed along the distal end 110B of the applicator 110, resulting in impedance that prevents flow through the dielectric material 111D. In the embodiment of FIGS. 7A and 7B, however, energy also passes through the portion 117B, so the energy is delivered in a pattern that includes the annular shape of the portion 117B. As a result, an energy pattern that would otherwise be generally limited to the same shape and dimension as gap 111C is now enlarged radially inward to an area including the annular area of portion 117B as shown in FIG. 7B. Where the annular thickness of the portion 117B is 2 mm as in the example above, the energy pattern is enlarged radially inward by 2 mm.

The application of sufficiently thick and thin layers of dielectric material 111D is not limited to the pattern shown in FIGS. 7A and 7B. For example, FIG. 7C illustrates an embodiment in which the dielectric layer 117 on the inner conductor 111B has a substantially uniform thickness, while the dielectric layer 116 on the outer conductor 111A is formed from the combination of portions 116A and 116B. As FIG. 7D illustrates, the layer 117 and the portion 116A are sufficiently thick to substantially prevent energy from being conducted through the layer 117 and the portion 116A, while the portion 116B is sufficiently thin to permit energy to pass to the eye. FIG. 7D shows another view of the surfaces of the layers 116 and 117 as indicated in FIG. 7C. As FIG. 7D illustrates, the thinner dielectric portion 116B defines a substantially annular shape that generally borders the annular gap 111C. Meanwhile, the thicker dielectric portion 116A defines a substantially annular shape that surrounds the annular portion 116B. When energy from the energy source 120 is conducted through the energy conducting element 111, energy can pass through the portion 116B, but not through the portion 116A. Accordingly, the energy pattern that would otherwise be generally limited to the same shape and dimension as gap 111C is now enlarged radially outward to an area including the annular area of portion 116B as shown in FIG. 7B. Thus, the embodiment of FIGS. 7C and 7D demonstrates that the layer 116 can also be configured with varying thicknesses. Indeed, it is contemplated that both layers 116 and 117 can be configured in the manner shown in FIGS. 7A-D to define an energy pattern that extends both radially inward and outward from the gap 111C.

The embodiments of FIGS. 7A-D illustrate energy patterns that are generally concentric with the outer conductor 111A and the inner conductor 111B and symmetric about the X- and Y-axes. However, as described previously, to treat an eye disorder, such as astigmatism, it may be necessary to deliver energy to the cornea in an irregularly shaped, e.g., asymmetric and/or non-annular, pattern. Accordingly, FIG. 7E illustrates an embodiment in which the dielectric layer 117 is applied to the inner conductor 111B to produce a non-annular and asymmetric pattern for delivering energy to selected areas of the cornea to treat the eye disorder. In particular, the dielectric layer 117 includes a thicker portion 117A and a thinner portion 117B. Unlike the embodiment of FIGS. 7A and 7B, however, the thicker portion 117A is not concentric with the inner conductor 111B or the gap 111C and the thinner portion 117B is non-annular. Moreover, the thicker portion 117A is not necessarily circular in shape. Because energy is delivered through the thinner portion 117B but not through the thicker portion 117A, the pattern for energy delivery to the eye includes the shape of the thinner portion 117B and is thus made non-annular and asymmetric.

The dielectric layer 116 applied to the FIGS. 7E and 7F is of sufficient thickness to prevent energy from passing through the entire layer 116. However, FIGS. 7G and 7H illustrate an alternative embodiment in which the dielectric layer 116 also includes a thicker portion 116A and a thinner portion 116B. In this alternative embodiment, energy is delivered through the thinner portions 116B and 117B but not through the thicker portions 116A and 117A. As a result, the pattern for energy delivery to the eye includes the shape of the thinner portions 116B and 117B. Thus, in contrast to the FIGS. 7E and 7F, the outer boundary for the delivery of energy extends beyond the substantially circular inner surface of the outer conductor 111.

In general, the inner and outer boundaries for the delivery of energy can be determined by employing dielectric layers of varying thickness, i.e., varying impedance, on the inner conductor 111B and the outer conductor 111A, respectively. Accordingly, the shapes for energy delivery shown in FIGS. 2B-Q can also be achieved by appropriate arrangement of thicker portions and thinner portions of dielectric material 111D on the outer conductor 111A and/or the inner conductor 111B. For example, FIG. 7I illustrates an arrangement of thicker portions 116A and 117A and thinner portions 116B and 117B that enables energy to be delivered from the applicator 110 in an elliptical shape defined by the gap 111C and the thinner portions 116B and 117B. Like the gap 111C formed in the embodiment of FIG. 2E, the energy is applied in a non-annular shape with substantially even thickness. It is contemplated that, similar to FIGS. 2F and 2G, the inner conductor 111B may be positioned non-concentrically with respect to the outer conductor 111A, so that the energy is also applied according to an asymmetric shape.

In another example, FIG. 7J illustrates how any appropriate combination of indentations and/or protrusions of varying shapes can also be produced by an arrangement of thicker portions 116A and 117A and thinner portions 116B and 117B on the outer conductor 111A and the inner conductor 111B, respectively. In particular, the thicker portion 116A and the thinner portion 116B define a curved protrusion 116C and a curved indentation 116D, while the thicker portion 117A and the thinner portion 117B define a notch-like protrusion 117C and a notch-like indentation 117D. The protrusions 116C and 117C extend inwardly from the gap 111C into the energy pattern delivered by the energy conducting element 111, while the indentations 116D and 117D extend outwardly from the gap 111C. Of course, embodiments are not limited to the specific combination, positions, shapes, and sizes of the indentations and protrusions 116C, 116D, 117C, and 117D shown in FIG. 7J.

FIG. 7K illustrates another technique for applying a dielectric material 111D to the distal end 110B of the energy conducting element 111. In particular, the dielectric material 111D may be applied to the outer conductor 111A so that one or more thicker portions 116A of the layer 116 creates intervals 111N similar to those shown in FIG. 2R. In addition, the dielectric material 111D may be applied to the inner conductor so that one or more thicker portions 117A creates interval 111O similar to those shown in FIG. 2R. The intervals 111N extend radially across the wall of the outer conductor 111A at the distal end 110B. Meanwhile, the intervals 111O extend across the inner conductor 111B. The intervals 111N and 111O have the effect of segmenting the outer conductor and inner conductor, respectively. Energy is conducted from areas of the gap 111C where the thicker portions 117B of the inner conductor 111B are opposed by the thicker sections 17A of outer conductor 111A. In other words, no energy is conducted from areas of the gap 111C that are positioned between the intervals 111N and the inner conductor 111B or between the intervals 111O and the outer conductor 111A. Thus, whereas FIGS. 7B and 7A illustrate embodiments that deliver energy in a continuous annular pattern defined by the annular gap 111C, the selected positioning of intervals 111N and 111O creates a non-continuous and segmented pattern in the embodiment of FIG. 7K. Of course, the embodiment shown in FIG. 7K is provided merely as an example, and any number of intervals 111N having any size may be employed to achieve a non-annular and/or asymmetric pattern. In addition, alternative embodiments may employ just the intervals 111N or just the intervals 111O, rather than both.

FIGS. 7A-K generally illustrate an outer conductor 111A and an inner conductor 111B that have substantially circular profiles. Embodiments employing varying thicknesses of a dielectric material 111D are not limited to energy conducting elements 111 with the shape profiles shown in FIGS. 7A-K. Indeed, the varying shapes and configurations for the outer conductor 111A and inner conductor 111B shown in FIGS. 2B-R may be combined with the various configurations of dielectric layers described herein. For example, FIG. 7L illustrates an energy conducting element 111 including a substantially elliptical outer conductor 111A in combination with a substantially cylindrical inner conductor 111B. As shown in FIG. 7L, the outer conductor 111A includes a thicker dielectric layer 116, while the inner conductor 111B has a dielectric layer 117 including a thicker portion 117A and a thinner portion 116B. The thicker portion 117A is substantially elliptical. As energy can pass through the remaining area of the dielectric layer 117 defined by the thinner portion 117B, the inner conductor 111B in effect behaves like an elliptically shaped inner conductor, e.g., similar to the inner conductor 111B of FIGS. 2E-G. In other embodiments, the dielectric layer 116 may also be further defined by a thicker dielectric portion and a thinner dielectric portion. Furthermore, embodiments are not limited to the arrangement of dielectric portions 117A and 117B shown in FIG. 7L.

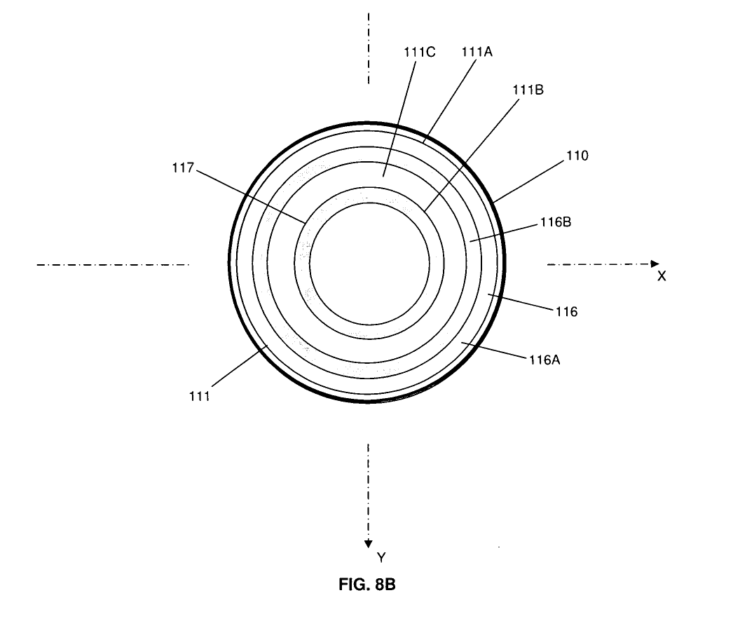

As described previously, thicker portions 116A and 117A and thinner portions 116B and 117B are combined to provide dielectric layers 116 and 117 that have varying impedance. In particular, the portions 116A and 117A must be thicker than the portions 116B and 117B if the same dielectric material 111D is employed for all portions 116A, 116B, 117A, and 117B, as impedance for a given material increases with thickness. However, it is contemplated that different dielectric materials 111D may be employed for different portions of the layers 116 and 117. As such, embodiments may employ layers 116 and 117 of substantially uniform thickness, but may have different portions of varying impedance. For example, FIG. 8A illustrates dielectric layers 116 and 117, each having substantially uniform thickness. However, the dielectric layer 116 in FIG. 8A includes portions 116A and 116B while dielectric layer 117 includes portions 117A and 117B. Although the portions 116A and 117A may have substantially the same thickness as 116B and 117B, respectively, the portions 116A and 117A provide higher impedance because they are formed from a dielectric material that has higher impedance for a given thickness when compared to the dielectric material of portions 116B and 117B, respectively. The impedance of portions 116A and 117A is sufficiently high to prevent passage of energy through the portions 116A and 117A. Meanwhile, the impedance of portions of 116B and 117B is sufficiently low to enable passage of energy through the layers 116B and 117B. As shown in FIG. 8B, the delivery of energy from the energy conducting element 111 extends from the gap 111C to the annular areas of portions 116B and 117B. Accordingly, the arrangement of different impedances according to portions 116A, 116B, 117A, and 117B shown in FIGS. 7F and 7H-L may be achieved by utilizing different dielectric materials for the portions, while providing different thickness profiles, e.g., keeping the thicknesses generally uniform, in some embodiments.

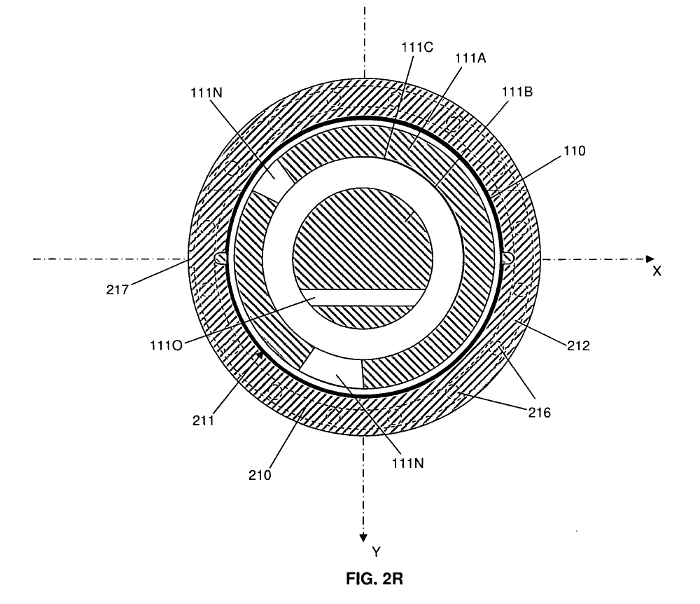

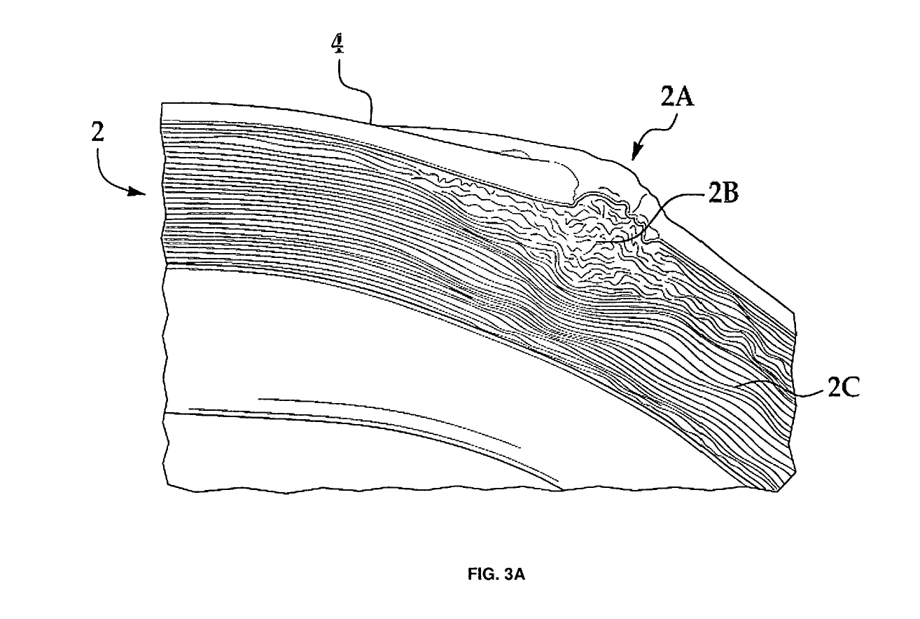

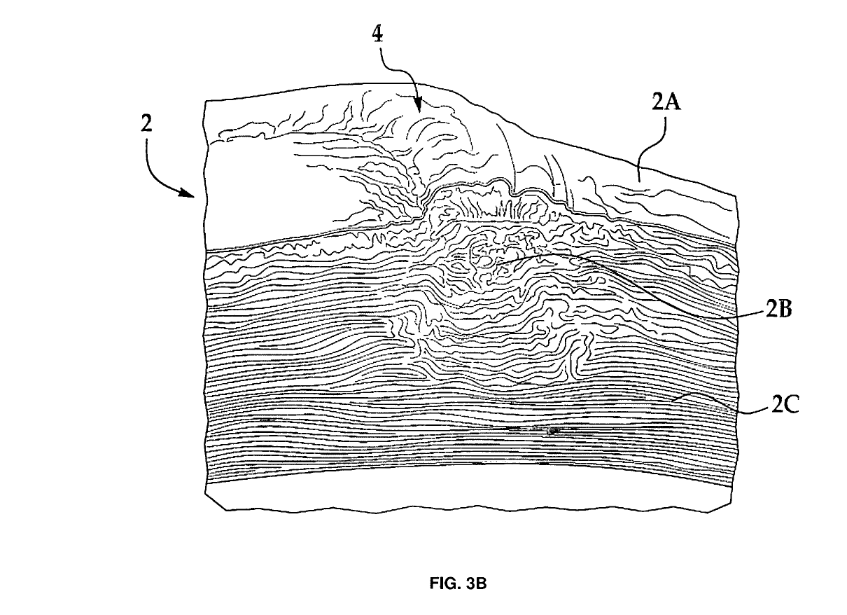

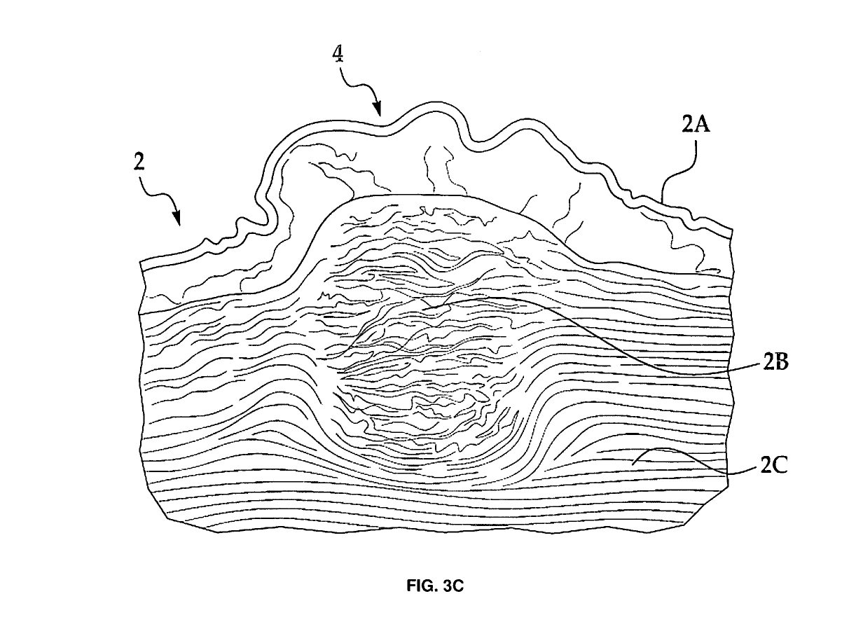

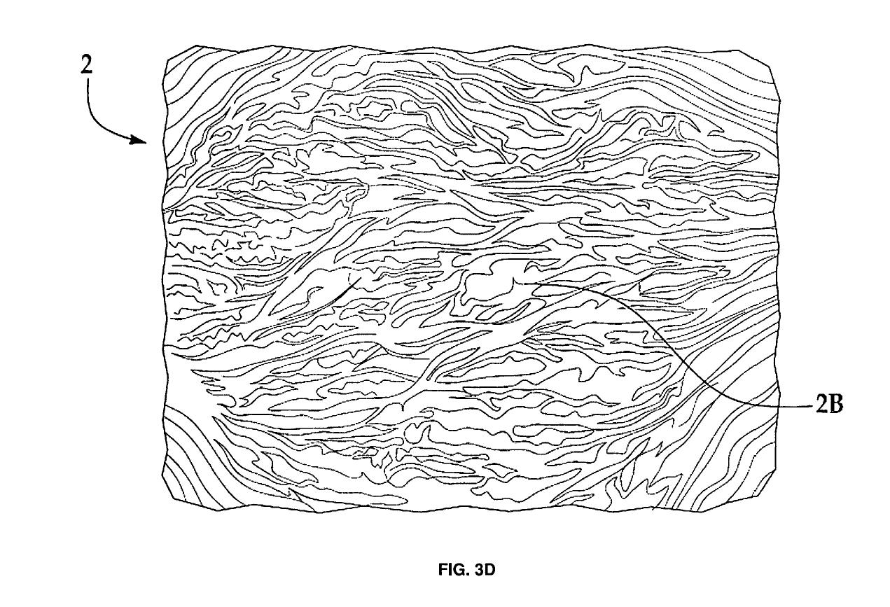

FIGS. 3A-D illustrate an example of the effect of applying energy to corneal tissue with a system for applying energy, such as the system illustrated in FIG. 1 and configured as described with reference to the exemplary embodiments illustrated in FIGS. 2A-2Q. In particular, FIGS. 3A and 3B illustrate high resolution images of the cornea 2 after energy has been applied. As FIGS. 3A and 3B show, a lesion 4 extends from the corneal surface 3A to a mid-depth region 3B in the corneal stroma 2D. The lesion 4 is the result of changes in corneal structure induced by the application of energy as described above. These changes in structure result in an overall reshaping of the cornea 2. It is noted that the application of energy, however, has not resulted in any energy-related damage to the corneal tissue.

As further illustrated in FIGS. 3A and 3B, the changes in corneal structure are localized and limited to an area and a depth specifically determined by an applicator as described above. FIGS. 3C and 3D illustrate histology images in which the tissue shown in FIGS. 3A and 3B has been stained to highlight the structural changes induced by the energy. In particular, the difference between the structure of collagen fibrils in the mid-depth region 2B where energy has penetrated and the structure of collagen fibrils outside the region 2B is clearly visible. Thus, the collagen fibrils outside the region 2B remain generally unaffected by the application of energy, while the collagen fibrils inside the region 2B have been rearranged and form new bonds to create completely different structures. In sum, the corneal areas experience a thermal transition to achieve a new state.

It has been discovered that as the corneal fibrils experience this thermal transition, there is a period in which the cornea also exhibits a plastic behavior, where the corneal structure experiences changes that make the cornea more susceptible to deformation by the application of additional mechanical forces. Therefore, embodiments may employ a shaped applicator 110 that applies an external molding pressure to the cornea 2, while the cornea 2 is reshaped with the shrinkage of corneal fibers in response to the application of energy during thermokeratoplasty.

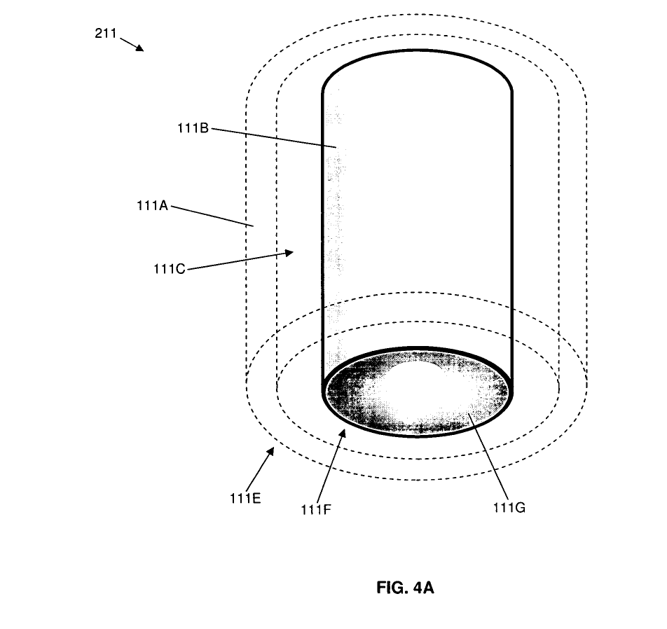

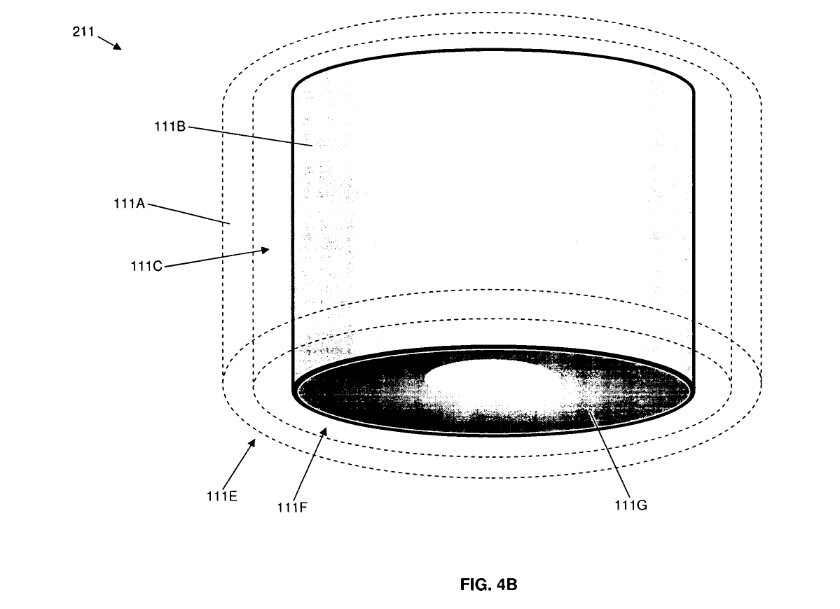

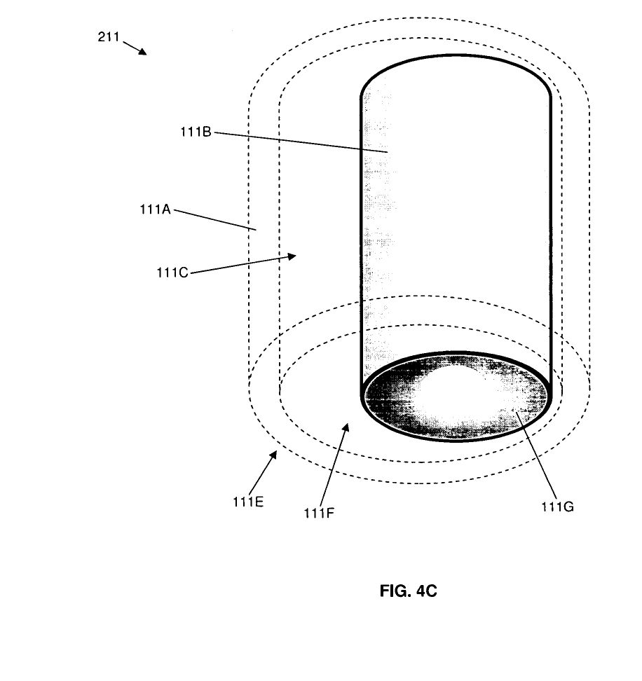

Accordingly, as illustrated in FIG. 1, the distal end 110B of the applicator 110 is configured to apply a molding pressure, or compression, to the corneal surface 2A and reshape the cornea 2 as the corneal structure experiences the state transition associated with the application of energy. As described previously, the energy conducting element 111 makes direct contact with the corneal surface 2A. FIG. 1 shows that the distal end 111F of the inner conductor 111B is in contact with the corneal surface 2A. Specifically, as is shown in FIGS. 4A-4C, the distal end 111F has a surface 111G which is concave and forms a mold over the center portion 2C of the cornea 2. FIGS. 4A-4C highlight the exemplary inner conductors 111B according to aspects of the present invention. In addition, FIGS. 4A-4C illustrate that surface 111G preferably retains a generally concave shape regardless of the size, shape, or position of inner conductor 111B.

During operation of the energy conducting element 111, the surface 111G is placed into contact with the portion 2C of the cornea 2 to apply molding pressures to the cornea 2. The amount of pressure applied by the surface 111G to an area of the corneal portion 2C depends on the shape of the surface 111G. For a given area of contact between the surface 111G and the portion 2C of the cornea, a greater pressure is exerted by the corresponding section of the surface 111G as the section extends farther against the cornea 2. As such, a particular shape for the surface 111G is selected to apply the desired molding profile. In particular, the surface 111G may be shaped to apply pressure in a non-annular and/or asymmetric profile to promote the treatment of astigmatism or other eye disorders as described previously. Thus, the reshaping of the cornea may depend on the combination of the shape of the gap 111C, the application of the dielectric layer 111C, and/or the shape of the surface 111G.

While the surface 111G may be integrally formed on the inner conductor 111B, the surface 111G may also be formed on an application end piece 111I, as shown in FIG. 1, that can be removably attached to the rest of the inner conductor 111B at the distal end 110B. As such, the surface 111G can be removed or changed. Advantageously, a variety of shapes for the surface 111G may be employed with a single inner conductor 111B by interchanging different end pieces 111I, each having a different corresponding surface 111G. In other words, instead of using a separate inner conductor 111B for each shape, a single energy conducting element 111 can accommodate different reshaping requirements. Furthermore, the end pieces 111I may be disposable after a single use to promote hygienic use of the applicator 110. The end piece 111I may be removably attached with the rest of the inner conductor 111B using any conductive coupling that still permits energy to be sufficiently conducted to the cornea 2. For example, the end piece 111I may be received via threaded engagement, snap connection, other mechanical interlocking, or the like.

The curvature of the surface 111G may approximate a desired corneal shape that will improve vision through the cornea 2. However, the actual curvature of the surface 111G may need to be greater than the desired curvature of the cornea 2, as the cornea 2 may not be completely plastic and may exhibit some elasticity that can reverse some of the deformation caused by the molding pressures. Moreover, as a flattening of the cornea 2 may be desired, the curvature of the surface 111G may also include flat portions. Accordingly, embodiments in general may employ a shaped surface 111G that achieves any type of reshaping. For example, embodiments may apply a shaped applicator to cause the cornea to be steepened or reshaped in an asymmetric fashion.

As described previously, some embodiments of the present invention do not maintain a fluid layer or a fluid flow between the energy conducting element 111 and the corneal surface 2A, thereby achieving a more predictable microwave pattern. Advantageously, in such embodiments, the molding pressures applied via the surface 111G are also more predictable as the contact between the surface 111G and the corneal area 2C is not affected by an intervening fluid layer or fluid flow.

As also described previously, the positioning system 200 places the distal end 110B of the applicator in a stable position over the cornea 2. As a result, the positioning system 200 may be employed to ensure that the surface 111G remains in contact with the corneal surface 2A and corresponding molding pressures are applied to the center portion 2C while energy is delivered via the energy conducting element 111. For example, as shown in FIG. 1, a coupling system 114 may be employed to couple the applicator 110 to the attachment element 210 of the positioning system 200. Once the applicator 110 is fully received into the attachment 210, the coupling system 114 prevents the applicator 110 from moving relative to the attachment element 210 along the Z-axis shown in FIG. 1. Thus, in combination with the attachment element 210, the energy conducting element 111, more particularly the surface 111G of the inner conductor 111B, can maintain its position against the corneal surface 2A and apply molding pressures to the center portion 2C of the cornea 2.

The coupling system 114 may include coupling elements 114A, such as tab-like structures, on the applicator 110 which are received into cavities 114B on the attachment element 210. As such, the coupling elements 114A may snap into engagement with the cavities 114B. The coupling elements 114A may be retractable to facilitate removal of the applicator 110 from the attachment element 210. For example, the coupling elements 114A may be rounded structures that extend from the applicator 110 on springs, e.g. coil or leaf springs (not shown). Additionally, the position of the coupling elements 114A along the Z-direction on the applicator 110 may be adjustable to ensure appropriate positioning of the applicator 110 with respect to the eye surface 2A and to provide the appropriate amount of molding pressure to the center portion 2C of the cornea 2.

It is understood, however, that the coupling system 114 may employ other techniques, e.g. mechanically interlocking or engaging structures, for coupling the applicator 110 to the attachment element 210. For example, the central passageway 211 of the attachment element 210 may have a threaded wall which receives the applicator 110 in threaded engagement. In such an embodiment, the applicator 110 may be screwed into the attachment element 210. The applicator can then be rotated about the Z-axis and moved laterally along the Z-axis to a desired position relative to the cornea 2.

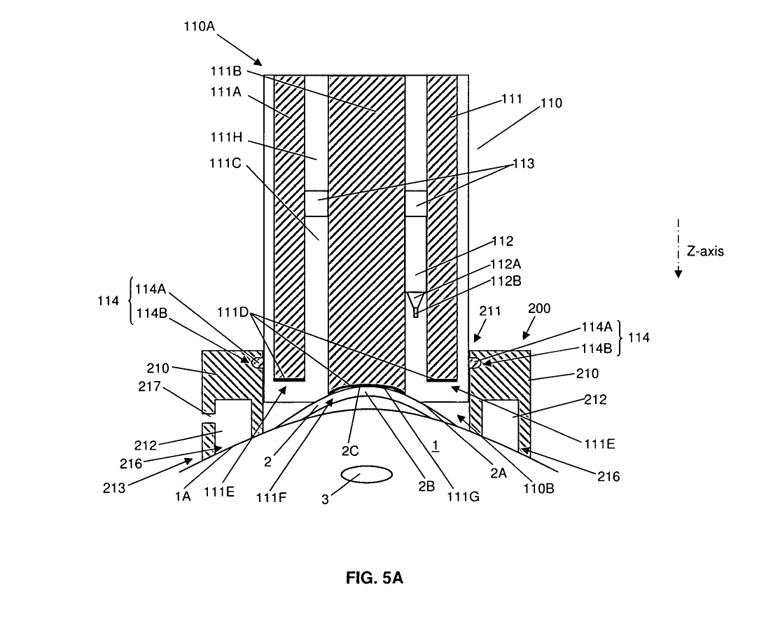

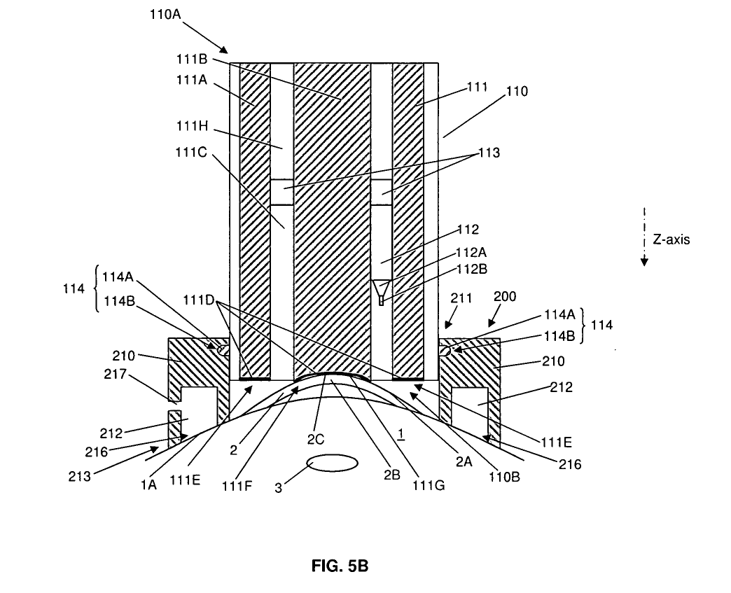

Although the distal end 111E of the outer conductor 111A shown in FIG. 1 extends past the distal end 111F of the inner conductor 111B, the position of the inner distal end 111F along the Z-axis is not limited to such a recessed position with respect to the outer distal end 111E. As shown in FIG. 5A, the inner distal end 111F may extend past the outer distal end 111E. Meanwhile, as shown in FIG. 5B, the inner distal end 111F and the outer distal end 111E extend to substantially the same position along the Z-axis.

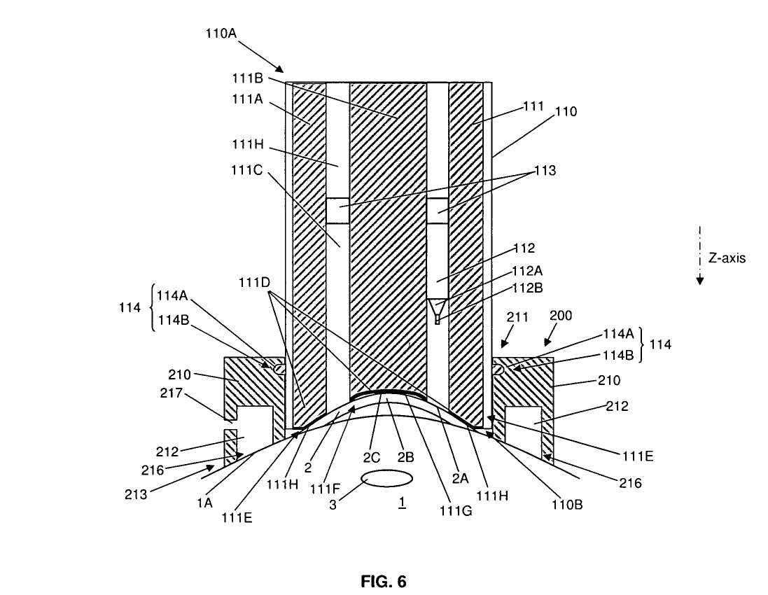

Additionally, as FIG. 6 illustrates, the distal end 111E of the outer conductor 111A may have a surface 111H that makes contact with the eye surface 1A. In some cases, the outer conductor 111A makes contact with the corneal surface 2A. Furthermore, the surface 111H may have a contoured surface that corresponds with the shape of the eye 1 where the surface 111H makes contact.

As described previously, the end piece 111I as shown in FIG. 1 may be disposable after a single use to promote hygienic use of the applicator 110. In general, the embodiments described herein may include disposable and replaceable components, or elements, to minimize cross-contamination and to facilitate preparation for procedures. In particular, components that are likely to come into contact with the patient's tissue and bodily fluids, such as the end piece 111I or even the entire applicator 110, are preferably discarded after a single use on the patient to minimize cross-contamination. Thus, embodiments may employ one or more use indicators which indicate whether a component of the system has been previously used. If it is determined from a use indicator that a component has been previously used, the entire system may be prevented from further operation so that the component cannot be reused and must be replaced.

For example, in the embodiment of FIG. 1, a use indicator 150 is employed to record usage data which may be read to determine whether the applicator 110 has already been used. In particular, the use indicator 150 may be a radio frequency identification (RFID) device, or similar data storage device, which contains usage data. The controller 130 may wirelessly read and write usage data to the RFID 150. For example, if the applicator 110 has not yet been used, an indicator field in the RFID device 150 may contain a null value. Before the controller 130 delivers energy from the energy source 120 to the energy conducting element 111, it reads the field in the RFID device 150. If the field contains a null value, this indicates to the controller 130 that the applicator 110 has not been used previously and that further operation of the applicator 110 is permitted. At this point, the controller 130 writes a value, such as a unique identifier associated with the controller 130, to the field in the RFID device 150 to indicate that the applicator 110 has been used. When a controller 130 later reads the field in the RFID device 150, the non-null value indicates to the controller 130 that the applicator 110 has been used previously, and the controller will not permit further operation of the applicator 110. Of course, the usage data written to the RFID device 150 may contain any characters or values, or combination thereof, to indicate whether the component has been previously used.

In another example, where the applicator 110 and the positioning system 200 in the embodiment of FIG. 1 are separate components, use indicators 150 and 250 may be employed respectively to indicate whether the application 110 or the positioning system 200 has been used previously. Similar to the use indicator 150 described previously, the use indicator 250, for example positioned on the attachment element 210, may be an RFID device which the controller 130 accesses wirelessly to read or write usage data. Before permitting operation of the applicator 110, the controller 130 reads the use indicators 150 and 250. If the controller 130 determines from the use indicators 150 and 250 that the applicator 110 and/or the positioning system 200 has already been used, the controller 130 does not proceed and does not permit further operation of the applicator 110. When the applicator 110 and the positioning system 200 are used, the controller 130 writes usage data to both use indicators 150 and 250 indicating that the two components have been used.

As described above with reference to FIGS. 7A and 8A for example, the distal end 111E of the outer conductor 111A and/or the distal end 111F of the inner conductor 111B may include applications of one or more dielectric materials 111D that provide varying impedance. The arrangement of areas of higher and lower impedance determines the pattern by which energy is delivered from the energy conducting element 111 to the eye. As FIGS. 7A and 8A also illustrate, the distal ends 111E and 111F may be provided on an end piece 111 that is removably attached to the rest of the energy conducting element 111. The end piece 111I may be removably attached using any conductive coupling that permits energy to be sufficiently conducted to the distal ends 111E and 111F. For example, the end piece 111I may be received via threaded engagement, snap connection, other mechanical interlocking, or the like. As shown in the FIGS. 7A and 8A, the end piece 111I may include both lower portions of the outer conductor 111A and inner conductor 111B coupled by a dielectric material 111H.

In addition to facilitating hygienic use of the applicator 110, removable end pieces 111I with varying applications of one or more dielectric materials may be employed to enable a single system to deliver energy to the eye according to different patterns. Advantageously, the use of such removable pieces 111I in effect allows the geometries of the applicator 110 to be modified without requiring physical modification of the shapes and configuration of the outer conductor 111A and the inner conductor 111B. For example, referring to FIGS. 7A and 7B, the inner conductor may have a diameter of approximately 7 mm. However, it may be determined that the energy must be applied according to an annular shape that extends the gap 111C inwardly by 2 mm. In other words, an inner conductor 111B having a diameter of 5 mm is desired. Rather than physically replacing the inner conductor 111B, an operator may implement an end piece 111I having a dielectric layer 117 with two portions 117A and 117B. In particular, the circular portion 117A would be concentric with the inner conductor 111B and have a diameter of 5 mm, while the annular portion 117B would surround the circular portion 117A and have an annular thickness of 2 mm. Because the circular portion 117A has high impedance and the annular portion 117B has low impedance, energy can be conducted through the annular portion 117B in addition to the gap 111C. As such, the dielectric layer 117 in effect creates an inner conductor 111B with a 5 mm diameter and a gap 111C that extends radially inward by 2 mm, thereby delivering energy to eye according to the desired geometries.

While various embodiments in accordance with the present invention have been shown and described, it is understood that the invention is not limited thereto. In particular, the treatment of astigmatism with embodiments of the present invention is described herein to illustrate, by way of example, various aspects of the present invention. It is understood, however, that the embodiments are not limited to the treatment of astigmatism and may be applied in similar manner to treat other eye disorders, particularly those involving asymmetric or irregular shaping of the cornea.

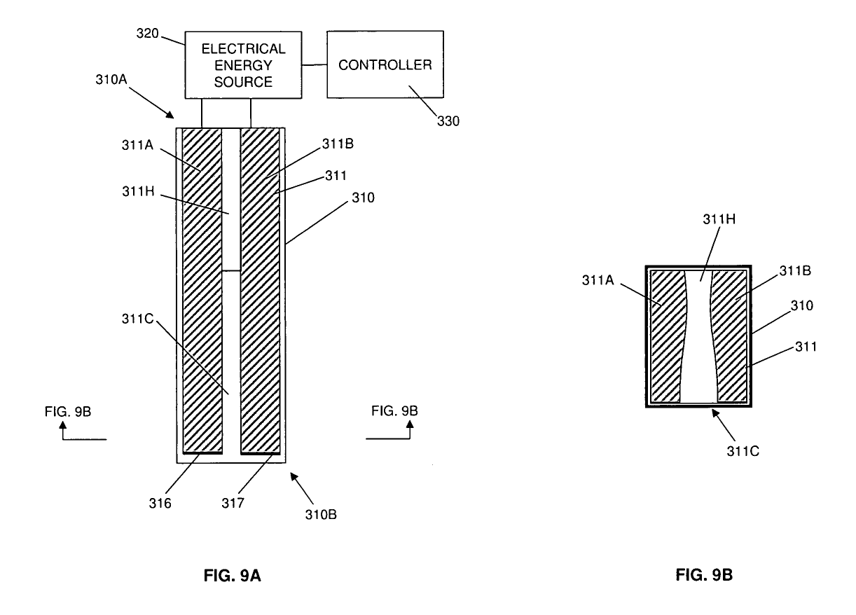

Although the embodiments described in detail herein may include an outer conductor 111A and an inner conductor 111B positioned therein, it is contemplated that conductors according to aspects of the present invention are not limited to this particular shape or configuration. The energy can be delivered by any configuration of opposing conductors. For example, FIG. 9A illustrates an applicator 310 including an energy conducting element 311 with two opposing conductor plates 311A and 311B. The energy conducting element 311 is operably connected to an electrical energy source 320 and a controller 330. The conductor plates 311A and 311B conduct energy from a proximal end 310A to a distal end 310B and applies energy to an eye according to the shape of the gap 311C. While the conductor plates 311A and 311B may be substantially planar and substantially parallel to each other, FIG. 9B shows that the conductor plates 311A and 311B may be selectively shaped to define a gap 311C that is non-planar and/or contoured on opposing sides. The energy conducting element 311 can apply energy to selected portions in asymmetric, as well as non-annular, patterns. It is contemplated that the teachings described herein, e.g., applying one or more dielectric layers 316, 317 of varying thickness, may be implemented with the conductors of FIG. 9 as well as conductors having other shapes and/or configurations.

As described previously, the positioning system 200 is employed to determine the position of the energy conducting element 111 relative to the eye. It is contemplated that, additionally or alternatively, the application of energy in an irregular pattern may be achieved through the selective positioning of the energy conducting element 111 with the positioning element 200. For example, asymmetry is determined with respect to features of the eye, so energy can be applied asymmetrically by positioning a symmetric energy conducting element 111 so that the center of the energy conducting element is offset from a center of an eye feature, e.g., the cornea. In general, the positioning system 200 receives the energy conducting element 111 and positions the distal end 110B relative to a feature of an eye. Based in part on the position of the energy conducting element 111, the gap 111C provides a pattern by which energy is delivered to the eye, where the pattern is non-annular and/or asymmetric with respect to the eye feature.

Furthermore, the present invention may be changed, modified and further applied by those skilled in the art. For example, although the applicator 110 in the examples above may be a separate element received into the positioning system 200, the applicator 110 and the positioning system 200 may be combined to form a more integrated device. Additionally, although the attachment element 210 in the embodiments above may be a vacuum device which is auctioned to the eye surface, it is contemplated that other types of attachment elements may be employed. For instance, the attachment element may be fixed to other portions of the head. Therefore, this invention is not limited to the detail shown and described previously, but also includes all such changes and modifications.

It is also understood that the figures provided in the present application are merely illustrative and serve to provide a clear understanding of the concepts described herein. The figures are not “to scale” and do not limit embodiments to the specific configurations and spatial relationships illustrated therein. In addition, the elements shown in each figure may omit some features of the illustrated embodiment for simplicity, but such omissions are not intended to limit the embodiment.

What is claimed is:

1. An energy conducting system for applying therapy to an eye, the energy conducting system comprising:
an energy conducting element including a first conductor and a second conductor, the first conductor and the second conductor extending to an application end and being separated by a gap; and
a positioning system receiving the energy conducting element and positioning the application end relative to a feature of an eye, the gap between the first conductor and the second conductor providing a pattern by which energy is delivered to the eye, the pattern being at least one of non-annular and asymmetric with respect to the eye feature.

2. The energy conducting system of claim 1, wherein the gap has a varying thickness defined by more than one distance between the first conductor and the second conductor.

3. The energy conducting system of claim 1, wherein the gap has a substantially constant thickness defined by substantially one distance between the first conductor and the second conductor.

4. The energy conducting system of claim 1, wherein the gap is substantially non-annular.

5. The energy conducting system of claim 1, wherein the gap is asymmetric relative to at least one transverse axis.

6. The energy conducting system of claim 1, wherein the gap is defined at least by an indentation that extends into at least one of the first conductor and the second conductor.

7. The energy conducting system of claim 6, wherein the indentation is a notch.

8. The energy conducting system of claim 6, wherein the indentation is curved.

9. The energy conducting system of claim 1, wherein the gap is defined at least by a protrusion that extends from at least one of the first conductor and the second conductor.

10. The energy conducting system of claim 9, wherein the protrusion has an angled shape.

11. The energy conducting system of claim 9, wherein the protrusion is curved.

12. The energy conducting system of claim 1, wherein the pattern is segmented according to at least one of the first conductor and the second conductor being segmented into more than two sections.

13. The energy conducting system of claim 12, wherein at least one of the first conductor and the second conductor are segmented into more than two sections according to a layer of at least one dielectric material, the layer providing varying impedance.

14. The energy conducting system of claim 1, wherein the application end includes a layer of at least one dielectric material, the layer providing varying impedance.

15. The energy conducting system of claim 1, wherein the first conductor and the second conductor are substantially planar and parallel to each other.

16. The energy conducting system of claim 1, wherein the first conductor is an outer conductor having an interior surface defining a longitudinal interior passageway; and the second conductor is an inner conductor positioned within the interior passageway of the outer conductor.

17. The energy conducting system of claim 16, wherein the inner conductor has an exterior surface separated from the outer conductor by a non-annular gap.

18. The energy conducting system of claim 16, wherein the inner conductor has an exterior surface separated from the interior surface of the outer conductor by the gap, the gap having a varying thickness defined by more than one distance between the exterior surface of the inner conductor and the interior surface of the outer conductor.

19. The energy conducting system of claim 1, wherein the application end includes an eye contact portion configured to apply the energy to an eye feature and providing a reshaping mold to reshape the eye feature as the eye feature responds to the application of the energy.

20. The energy conducting system of claim 1, wherein the positioning system comprises a vacuum fixation device creating a vacuum connection with an eye and to position the energy conducting element relative to the eye, whereby the energy conducting element directs the energy to the eye.

21. The energy conducting system of claim 1, wherein the application end is one of a plurality of removably attachable end pieces.

22. An energy conducting system for applying therapy to an eye, the energy conducting system comprising:
an outer conductor having an interior surface defining an interior passageway; and
an inner conductor positioned within the interior passageway, the inner conductor having an exterior surface separated from the interior surface of the outer conductor by a gap, the gap being at least one of non-annular and asymmetric,
wherein the outer conductor and inner conductor define an application end positionable at an eye, the outer conductor and inner conductor conducting energy to the eye via the application end according to a pattern defined at least by the gap.

23. The energy conducting system of claim 22, wherein the inner conductor has an inner-conductor center axis that is offset from an interior-passageway center axis of the interior passageway.

24. The energy conducting system of claim 23, wherein the interior passageway and the inner conductor are substantially cylindrical.

25. The energy conducting system of claim 23, wherein the interior passageway and the inner conductor have transverse profiles that are substantially elliptical.

26. The energy conducting system of claim 23, wherein the inner-conductor center axis is adjustably movable relative to the interior-passageway center axis.

27. The energy conducting system of claim 22, wherein the gap between the inner conductor and the outer conductor has a transverse profile that is substantially elliptical.

28. The energy conducting system of claim 22, wherein the gap has a varying thickness defined by more than one distance between the outer conductor and the inner conductor.

29. The energy conducting system of 22, wherein the gap has a substantially constant thickness defined by substantially one distance between the first conductor and the second conductor.

30. The energy conducting system of claim 22, wherein the gap is defined at least by an indentation that extends into at least one of the outer conductor and the inner conductor.

31. The energy conducting system of claim 30, wherein the indentation is a notch.

32. The energy conducting system of claim 30, wherein the indentation is curved.

33. The energy conducting system of claim 22, wherein the gap is defined at least by a protrusion that extends from at least one of the first conductor and the second conductor.

34. The energy conducting system of claim 33, wherein the protrusion has an angled shape.

35. The energy conducting system of claim 33, wherein the protrusion is curved.

36. The energy conducting system of claim 22, wherein at least one of the outer conductor and the inner conductor being segmented into more than two sections.

37. The energy conducting system of claim 36, wherein at least one of the outer conductor and the inner conductor are segmented into more than two sections according to a layer of at least one dielectric material, the layer providing varying impedance.

38. The energy conducting system of claim 22, wherein the application end includes a layer of at least one dielectric material, the layer providing varying impedance.

39. The energy conducting system of claim 22, wherein the application end includes an eye contact portion configured to apply the energy to an eye feature and providing a reshaping mold to reshape the eye feature as the eye feature responds to the application of the energy.

40. The energy conducting system of claim 22, further comprising a positioning system receiving the outer conductor and the inner conductor and positioning the application end relative to a feature of an eye.

41. The energy conducting system of claim 40, wherein the positioning system comprises a vacuum fixation device creating a vacuum connection with an eye and to position the energy conducting element relative to the eye, whereby the energy conducting element directs the energy to the eye.

42. The energy conducting system of claim 22, wherein the application end is one of a plurality of removably attachable end pieces.

43. An energy conducting system for applying therapy to an eye, the energy conducting system comprising:
an energy conducting element including a first conductor and a second conductor, the first conductor and the second conductor extending to an application end and being separated by a gap; and
one or more materials providing varying impedance, the one or more materials being applied, at the application end, to at least one of the first conductor and the second conductor, the first conductor and the second conductor conducting energy to the eye via the application end at least according to a pattern defined by the varying impedance.

44. The energy conducting system of claim 43, wherein the one or more materials includes a material applied in layers of varying thickness to provide varying impedance.

45. The energy conducting system of claim 43, wherein the one or more materials includes materials that provide varying impedance for a given applied thickness.

46. The energy conducting system of claim 43, wherein the pattern for conducting energy is asymmetric.

47. The energy conducting system of claim 43, wherein the pattern for conducting energy is non-annular.

48. The energy conducting system of claim 43, wherein the pattern for conducting energy is substantially elliptical.

49. The energy conducting system of claim 43, wherein the varying impedance includes at least one area of high impedance preventing conduction of energy therefrom and at least one area of low impedance allowing conduction of energy therefrom, the areas of high impedance and the areas of low impedance defining the pattern for conducting energy via the application end.

50. The energy conducting system of claim 49, wherein the at least one area of low impedance includes at least one protrusion extending into the gap.

51. The energy conducting system of claim 49, wherein the at least one area of low impedance includes at least one indentation extending from the gap.

52. The energy conducting system of claim 43, wherein the gap is non-annular.

53. The energy conducting system of claim 43, wherein the gap is asymmetric.

54. The energy conducting system of claim 43, wherein the gap includes a protrusion.

55. The energy conducting system of claim 43, wherein the gap includes an indentation.

56. The energy conducting system of claim 43, wherein the first conductor is an outer conductor having an interior surface defining a longitudinal interior passageway; and the second conductor is an inner conductor positioned within the interior passageway of the outer conductor.

57. The energy conducting system of claim 56, wherein the one or more materials is applied to the inner conductor, the application of the one or more materials including a circular area of high impedance and an annular area of low impedance surrounding the circular area of high impedance.

58. The energy conducting system of claim 43, wherein the application end includes an eye contact portion configured to apply the energy to an eye feature and providing a reshaping mold to reshape the eye feature as the eye feature responds to the application of the energy.

59. The energy conducting system of claim 43, further comprising a positioning system receiving the energy conducting element and positioning the application end relative to a feature of an eye.

60. The energy conducting system of claim 59, wherein the positioning system comprises a vacuum fixation device creating a vacuum connection with an eye and to position the energy conducting element relative to the eye, whereby the energy conducting element directs the energy to the eye.

61. The energy conducting system of claim 43, wherein the application end is one of a plurality of removably attachable end pieces.

62. A method for applying therapy to an eye with a conducting system comprising an energy conducting element including a first conductor and a second conductor, the first conductor and the second conductor extending to an application end and being separated by a gap, and a positioning system receiving the energy conducting element, the method comprising the steps of:
determining a gap separating the first conductor and the second conductor;
positioning, with the positioning system, the application end of the energy conducting element at an eye; and
reshaping an eye feature by applying energy to the eye via the conducting element according to a pattern, the pattern being defined at least by the gap and the position of the application end relative to the eye and being at least one of non-annular and asymmetric with respect to the eye feature.

63. The method of claim 62, wherein the step of determining a gap comprises providing a gap with a varying thickness defined by more than one distance between the first conductor and the second conductor.

64. The method of claim 62, wherein the step of determining a gap comprises providing a gap with a substantially constant thickness defined by substantially one distance between the first conductor and the second conductor.

65. The method of claim 62, wherein the step of determining a gap comprises providing a gap that is substantially non-annular.

66. The method of claim 62, wherein the step of determining a gap comprises providing a gap that is asymmetric relative to at least one transverse axis.

67. The method of claim 62, wherein the step of determining a gap comprises providing a gap that is defined at least by an indentation that extends into at least one of the first conductor and the second conductor.

68. The method of claim 67, wherein the indentation is a notch.

69. The method of claim 67, wherein the indentation is curved.

70. The method of claim 62, wherein the step of determining a gap comprises providing a gap that is defined at least by a protrusion that extends from at least one of the first conductor and the second conductor.

71. The method of claim 70, wherein the protrusion has an angled shape.

72. The method of claim 70, wherein the protrusion is curved.

73. The method of claim 62, further segmenting at least one of the first conductor and the second conductor into more than two sections to further define the pattern.

74. The method of claim 73, the step of segmenting comprises applying a layer of at least one dielectric material to at least one of the first conductor and the second conductor, the layer providing varying impedance.

75. The method of claim 62, further comprises applying a layer of at least one dielectric material to at least one of the first conductor and the second conductor, the layer providing varying impedance.

76. The method of claim 62, wherein the step of determining a gap comprises providing a gap defined by the first conductor and the second conductor being substantially planar and parallel to each other.

77. The method of claim 62, wherein the first conductor is an outer conductor having an interior surface defining a longitudinal interior passageway; and the second conductor is an inner conductor positioned within the interior passageway of the outer conductor.

78. The method of claim 77, wherein the step of determining a gap comprises providing a non-annular gap between the inner conductor and the outer conductor.

79. The method of claim 77, wherein the step of determining a gap comprises providing a gap having a varying thickness defined by more than one distance between the exterior surface of the inner conductor and the interior surface of the outer conductor.

80. The method of claim 62, wherein the application end includes an eye contact portion configured to apply the energy to an eye feature and providing a reshaping mold to reshape the eye feature as the eye feature responds to the application of the energy.

81. The method of claim 62, wherein the positioning system comprises a vacuum fixation device creating a vacuum connection with an eye and to position the energy conducting element relative to the eye, whereby the energy conducting element directs the energy to the eye.

82. The method of claim 62, further comprising replacing the application end with one of a plurality of removably attachable end pieces.

83. A method for applying therapy to an eye with a conducting assembly comprising an energy conducting element including a first conductor and a second conductor, the first conductor and the second conductor extending to an application end and being separated by a gap; and one or more materials providing varying impedance, the one or more materials being applied, at the application end, to at least one of the first conductor and the second conductor, the first conductor and the second conductor conducting energy to the eye via the application end at least according to a pattern defined by the varying impedance, the method comprising:
positioning the application end of the conducting assembly at an eye; and
reshaping an eye feature by applying energy to the eye via the conducting element according to the varying impedance. reshaping an eye feature by applying energy to the eye via the conducting element according to a pattern, the pattern being defined at least by the one or more materials and the position of the application end relative to the eye feature.

84. The method of claim 83, wherein the one or more materials includes a material applied in layers of varying thickness to provide varying impedance.

85. The method of claim 83, wherein the one or more materials includes materials that provide varying impedance for a given applied thickness.

86. The method of claim 83, wherein the pattern for conducting energy is asymmetric.

87. The method of claim 83, wherein the pattern for conducting energy is non-annular.

88. The method of claim 83, wherein the pattern for conducting energy is substantially elliptical.

89. The method of claim 83, wherein the varying impedance includes at least one area of high impedance preventing conduction of energy therefrom and at least one area of low impedance allowing conduction of energy therefrom, the areas of high impedance and the areas of low impedance defining the pattern for conducting energy via the application end.

90. The method of claim 89, wherein the at least one area of low impedance includes at least one protrusion extending into the gap.

91. The method of claim 89, wherein the at least one area of low impedance includes at least one indentation extending from the gap.

92. The method of claim 83, wherein the gap is non-annular.

93. The method of claim 83, wherein the gap is asymmetric.

94. The method of claim 83, wherein the gap includes a protrusion.

95. The method of claim 83, wherein the gap includes an indentation.

96. The method of claim 83, wherein the first conductor is an outer conductor having an interior surface defining a longitudinal interior passageway; and the second conductor is an inner conductor positioned within the interior passageway of the outer conductor.

97. The method of claim 83, wherein the positioning system comprises a vacuum fixation device creating a vacuum connection with an eye and to position the energy conducting element relative to the eye, whereby the energy conducting element directs the energy to the eye.

98. The method of claim 83, further comprising replacing the application end with one of a plurality of removably attachable end pieces.
